(19) United States
(12) Patent Application Publication
(10) Pub. No.: US 2020/0157609 A1
(43) Pub. Date: May 21, 2020

(54) SALIVARY AND GUT MICROBIOTA TO DETERMINE COGNITIVE INPAIRMENT

(71) Applicants: Virginia Commonwealth University, Richmond, VA (US); George Mason University, Fairfax, VA (US)

(72) Inventors: Jasmohan Bajaj, Richmond, VA (US); Patrick M. Gillevet, Fairfax, VA (US)

(21) Appl. No.: 16/659,915

(22) Filed: Oct. 22, 2019

Related U.S. Application Data
(60) Provisional application No. 62/748,809, filed on Oct. 22, 2018.

(51) Int. Cl. C12Q 1/689

(52) U.S. Cl. CPC C12Q 1/689

(57) ABSTRACT

Methods of detecting and treating cognitive impairment are provided. The methods involve determining, via a metagenomic analysis, the salivary and/or gut microbiota signature of the subject, e.g. a subject with an illness such as chronic liver disease and/or post-traumatic stress disorder (PTSD), and based in the microbiota signature, identifying subjects who have cognitive impairment.

### CROSS REFERENCE TO RELATED APPLICATIONS

This application claims the benefit of U.S. Provisional Application No. 62/748,809, filed Oct. 22, 2018.

### STATEMENT REGARDING FEDERALLY SPONSORED RESEARCH OR DEVELOPMENT

This invention was made with government support under Grant Numbers: 2I0CX001076 and R21TR002024 awarded by the United States Department of Veterans Affairs. The government has certain rights in the invention.

### BACKGROUND OF THE INVENTION

### Field of the Invention

This invention generally relates to the detection and treatment of cognitive impairment. In particular, the invention relates to the detection of covert cognitive impairment in a subject by determining the salivary and/or gut microbiota signature of the subject.

### State of Technology

The cognitive or brain-related burden of chronic diseases is increasing in the population. As a result, people are living longer but are not living better. Complaints of cognitive issues are very frequent for patients with leading chronic diseases such as liver diseases, morbid obesity, diabetes and chronic renal failure, but it is difficult to diagnose these conditions due to imprecise testing methods and logistical issues.

Hepatic encephalopathy (HE) is one of the leading causes of morbidity and mortality in cirrhotic patients (1, 2). The spectrum of neuro-cognitive impairment in cirrhosis ranges from the subtle covert HE (CHE) through disorientation, stupor and coma known as overt

HE (OHE) (2). Covert HE, which includes minimal HE (MHE), is associated with medical outcomes such as progression to OHE, hospitalizations and death; as well as psycho-social outcomes such as impaired driving ability, a lower health-related quality of life and socio-economic status (3). MHE and CHE are used inter-changeably throughout the document and in the literature. Therefore, the diagnosis of CHE is important but is rarely made because CHE diagnostic tests are often associated with poor inter-test agreement (4-7). In addition, testing strategies for CHE interrogate different brain regions and are differentially associated with ammonia and systemic inflammation (8-10).

Underlying this spectrum of cognitive impairment is an altered gut-liver-brain axis. The components of these alterations include an unfavorable gut microbiota composition, increased local and systemic inflammation, and impaired immune response (11-13). However, to date there has been limited study of the association between gut microbiota profiles with differing CHE testing strategies and the potential practical use of specific microbial profiles to diagnose CHE.

### SUMMARY OF THE INVENTION

The disclosure provides gut and salivary microbial profiles (signatures) associated with cognitive dysfunction of patients with a chronic disease. In some aspects, the cognitive dysfunction that is detected is covert cognitive dysfunction, e.g. dysfunction that is occurring or has occurred but has had minimal impairment in instrumental activities of daily living of the subject. The microbial signatures are advantageously obtained non-invasively from one or both of stool and saliva samples. As an example, the patients with cirrhosis and covert hepatic encephalopathy (CHE), defined according to specific extant cognitive assessment strategies, were shown to have unique microbial signatures in the stool (e.g. as a proxy for gut) and saliva. These microbial signatures were associated with the diagnosis of CHE independent of clinical criteria, and the presence of specific bacterial taxa was indicative of either CHE or normal cognition. As another example, the patients with cirrhosis also have PTSD, which predisposes them to/is associated with cognitive dysfunction, which is sometimes severe cognitive dysfunction.

Accordingly, the disclosure provides methods of determining the salivary and/or stool microbiota signature of a subject of interest, for example, a subject with an illness, such as a chronic illness, that may predispose the subject to cognitive impairment. The methods are non-invasive, simple, reliable, and cost effective point-of-care tests which evaluate brain function and permit the diagnosis, and thus the appropriate treatment, of subjects who are identified as having abnormal cognition, especially covert abnormal cognition. This enables implementation of early therapeutic measures to prevent or avoid the onset of more severe or overt cognitive impairment.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this invention to provide a metagenomic method of diagnosing cognitive impairment in a subject with cirrhosis, comprising collecting a stool sample and/or a saliva sample from the subject with cirrhosis; contacting at least a portion of the stool sample and/or at least a portion of the saliva sample with a substrate comprising nucleic acid sequences that bind to: for the stool sample, 16S rRNA of at least one of Veillonellaceae, Enterococcus, Proteobacteria, Ruminococcaceae and Lachnospiraceae; and for the saliva sample, 16S rRNA of at least one of Lactobacillaceae, Coriobacteriaceae, Clostridiales cluster XI, Aerococcaceae and Prevotellaceae; detecting the presence of microbes by sequencing 16S rRNA to which the nucleic acid sequences are bound; and diagnosing the subject as having cognitive impairment when for the stool sample, the presence of one or more of Enterococcus, Proteobacteria and Veillonellaceae is detected; and for the saliva sample, to the presence of one or more of the Lactobacillaceae, Coriobacteriaceae, Clostridiales cluster XI, Aerococcaceae and Prevotellaceae is detected. In some aspects, the step of collecting is performed by collecting the stool sample. In some aspects, the step of collecting is performed by collecting the saliva sample. In additional aspects, the nucleic acid sequences bind to 16S rRNA of Veillonellaceae, Enterococcus, Proteobacteria, Ruminococcaceae and Lachnospiraceae. In further aspects, the nucleic acid sequences bind to 16S rRNA of Lactobacillaceae, Coriobacteriaceae, Clostridiales cluster XI, Aerococcaceae and Prevotellaceae. In other aspects, the step of detecting is performed using one of more of the following: microarrays, qPCR and RNA sequencing. In yet further aspects, the step of detecting comprises the steps of amplifying and tagging the 16S rRNA by PCR with an amplification primer pair comprising: i) a high throughput sequencing adaptor, ii) a tag sequence to identify the originating sample, and iii) a priming sequence that is the same in each sample, the priming sequence hybridizing 3′ to a genetic region that is variable across microbial species; and sequencing the 16S rRNA, wherein 16S rRNA sequences are assigned to the originating sample by the nucleotide sequence of the tag sequence.

The disclosure also provides methods of detecting and treating cognitive impairment in a subject suffering from a chronic disease, comprising establishing a microbial signature for one or both of stool and saliva of the subject by detecting, metagenomically, the presence of at least one microbe in one or both of a stool sample and a saliva sample of the subject, comparing the microbial signature to at least one corresponding reference microbial signature, wherein the at least one corresponding reference microbial signature is a negative reference microbial signature obtained from subjects who are not suffering from covert cognitive impairment and/or a positive reference microbial signature obtained from subjects who are suffering from covert cognitive impairment; and treating a subject for cognitive impairment when the microbial signature differs from that of the negative reference microbial signature and/or is the same as that of the positive reference microbial signature; wherein the step of establishing is performed by identifying a subject having the chronic disease, collecting a stool sample and/or a saliva sample from the subject; and detecting microbes present in the stool sample and/or the saliva sample; and wherein the step of detecting is performed by sequencing 16S rRNA. In some aspects, the step of treating includes administering to the subject one or more of: rifaximin, lactulose a calcineurin inhibitor (CNI), omega 3 fatty acids, and a bacteriotherapy. In some aspects, the CNI is cyclosporine or tacrolimus. In other aspects, the chronic disease is cirrhosis, heart failure, chronic kidney disease, diabetes or chronic obstructive pulmonary disease. In additional aspects, the chronic disease is cirrhosis. In yet further aspects, the cognitive impairment is covert hepatic encephalopathy (CHE). In certain aspects, the microbial signature is established by in the stool sample, detecting the presence of at least one of Veillonellaceae and Lachnospiraceae; and/or in the saliva sample, detecting the presence of at least one of Lactobacillaceae, Coriobacteriaceae, Clostridiales cluster XI, Aerococcaceae and Prevotellaceae. In additional aspects, the presence of Veillonellaceae in the stool sample indicates that the subject has CHE and the presence of Lachnospiraceae indicates that the subject does not have CHE; and the presence of Lactobacillaceae and/or Coriobacteriaceae in the saliva sample indicates that the subject has CHE; and the presence of Clostridiales clusterXI, Aerococcaceae and/or Prevotellaceae indicates that the subject does not have CHE. In additional aspects, the bacteriotherapy includes administration of one or more of Lachnospiraceae, Clostridiales clusterXI, Aerococcaceae and/or Prevotellaceae. In some aspects, the subject has PTSD. In further aspects, the microbial signature is established by, in the stool sample, detecting the presence of at least one of Enterococcus, Proteobacteria, Ruminococcaceae and Lachnospiraceae. In some aspects, the presence of Enterococcus and/or Proteobacteria in the stool sample indicates that the subject has cognitive impairment and the presence of Ruminococcaceae and/or Lachnospiraceae indicates that the subject does not have cognitive impairment. In additional aspects, the bacteriotherapy includes administration of one or both of Ruminococcaceae and Lachnospiraceae.

### BRIEF DESCRIPTION OF THE DRAWINGS

FIG. 1A and B. Flowchart of patients. A, patients who had stool collected and were tested for CHE using the specific modalities; B, patients who had saliva collected and were tested for CHE. ICT: inhibitory control test, PHES: psychometric hepatic encephalopathy score.

FIG. 2A-C. Linear discriminant effect size (LEFSe) changes in stool of patients with and without covert hepatic encephalopathy, regardless of prior overt hepatic encephalopathy. Bars in dark gray represent bacterial taxa that were higher in patients who tested negative for CHE while those in gray represent bacterial taxa that were higher in patients who tested positive for CHE using the indicated modality. A, PHES; B, ICT; C, Stroop.

FIG. 3A-C. LEFSe changes in stool of patients with and without covert hepatic encephalopathy who had not had prior overt hepatic encephalopathy. Bars in dark gray represent bacterial taxa that were higher in patients who tested negative for CHE while those in gray represent bacterial taxa that were higher in patients who tested positive for CHE using the indicated modality. A, PHES; B, ICT; C, Stroop.

FIG. 4A-C. LEFSe changes in saliva of patients with and without covert hepatic encephalopathy, regardless of prior overt hepatic encephalopathy. Bars in dark gray represent bacterial taxa that were higher in patients who tested negative for CHE while those in gray represent bacterial taxa that were higher in patients who tested positive for CHE using the indicated modality. A, PHES; B, ICT; C, Stroop.

FIG. 5A-C. LEFSe changes in saliva of patients with and without covert hepatic encephalopathy who had not had prior overt hepatic encephalopathy. Bars in dark gray represent bacterial taxa that were higher in patients who tested negative for CHE while those in gray represent bacterial taxa that were higher in patients who tested positive for CHE using the indicated modality. A, PHES; B, ICT; C, Snoop.

FIG. 6A-C. Correlation network between microbiota and cognitive tests were performed using R, and significant interactions with P<0.05 and r>0.6 or <−0.6 are shown. Subnetworks centered around Enterococcus and Escherichia/Shigella are shown in A-C. Microbiota are presented as Family I Genus and are shown as oval nodes, while cognitive tests are shown as diamond nodes. Solid lines indicate negative, while broken lines indicate positive correlations. OHE, prior overt hepatic encephalopathy currently on lactulose and rifaximin; PTSD, posttraumatic stress disorder. A: no-OHE PTSD group showing negative correlations between Enterococcus and Escherichia/Shigella and several members of the Lachnospiraceae and Ruminococcaceae families B: OHE no-PTSD group showing Enterococcus associated negatively with several members of the Lachnospiraceae and Ruminococcaceae families Escherichia/Shigella were not meaningfully linked. C: OHE PTSD group showing negative correlations between Enterococcus and Escherichia/Shigella and several members of the Lachnospiraceae and Ruminococcaceae families Cognitive performance on lures (higher=worse) was linked positively with Enterococcus and negatively with Lachnospiraceae and Ruminococcaceae genera. The block design test (BDT; high=better) was positively linked with Lachnospiraceae and Ruminococcaceae genera and negatively with Escherichia/Shigella. The linkage was similar with the number connection test-A (NCT-A; high=worse) with positive correlation with Escherichia/Shigella and negatively with Lachnospiraceae and Ruminococcaceae genera.

FIG. 7A-D. Correlation network between microbiota and cognitive tests were performed using R, and significant interactions with P<0.05 and r>0.6 or <−0.6 are shown. Subnetworks centered round Fecalibacterium are shown in A-D. Microbiota are presented as Family I Genus and are shown in oval nodes, while cognitive tests are shown in diamond nodes. Solid lines indicate negative, while broken lines indicate positive correlations. OHE, prior overt hepatic encephalopathy currently on lactulose and rifaximin; PTSD, posttraumatic stress disorder. A: no-OHE no-PTSD group showed positive correlations between Fecalibacterium and other Lachnospiraceae and Ruminococcaceae genera. B: no-OHE PTSD group again showed positive correlations between Fecalibacterium and other Lachnospiraceae and Ruminococcaceae genera. Line tracing time (high=worse) was negatively correlated with Lachnospiraceae and Ruminococcaceae genera and positively with Fusobacterium. C: OHE no-PTSD demonstrated negative correlations with Fecalibacterium and Escherichia/Shigella, Enterococcus, Prevotella, and positive with other Lachnospiraceae and Ruminococcaceae genera. D: OHE PTSD group correlation network showed similar negative correlations between Fecalibacterium and Escherichia/Shigella, Enterococcus, Streptococcus, and Veillonella. Block design test (BDT high=better) was positively linked with Fecalibacterium. Positive linkages were demonstrated with other Lachnospiraceae and Ruminococcaceae genera.

FIG. 8. Schematic representation of a system for implementing the methods disclosed herein.

### DETAILED DESCRIPTION

Stool (as a proxy for gut) and salivary microbial signatures associated with cognitive dysfunction in subjects with a chronic disease and various conditions are disclosed herein. The signatures are obtained by metagnomic analysis, i.e. directly from the biological samples, without an intervening step of culturing the microbes in a sample. However, it is noted that culturing of one or more of the microbes can be undertaken, if desired. The signatures are used in methods of diagnosing cognitive dysfunction and are especially helpful to diagnose covert cognitive dysfunction in subjects so that early treatments can be provided, e.g. to reverse the symptoms, and/or so that progression to overt cognitive dysfunction can be avoided. Since the samples that are assessed are stool and/or saliva, the methods are advantageously non-invasive.

As an example, the presence of specific microbes in stool and saliva samples were shown to correlate with a diagnosis of CHE in subjects suffering from cirrhosis, the diagnosis being confirmed using standard known test methods. For this group of subjects, at a minimum, the microorganisms that are detected in stool samples include at least one of Veillonellaceae and Lachnospiraceae; and the microorganisms that are detected in saliva samples include at least one of Lactobacillaceae, Coriobacteriaceae, Clostridiales cluster XI, Aerococcaceae and Prevotellaceae.

In particular:
I. in subjects with no prior diagnosis of overt cognitive dysfunction, the presence of Veillonellaceae in a stool sample indicates that the subject has covert cognitive dysfunction and the presence of Lachnospiraceae indicates that the subject does not have covert cognitive dysfunction; and the presence of Lactobacillaceae and/or Coriobacteriaceae in a saliva sample indicates that the subject has covert cognitive dysfunction; and the presence of Clostridiales clusterXI, Aerococcaceae and/or Prevotellaceae indicates that the subject does not have covert cognitive dysfunction; and
II. in subjects with a previous diagnosis of overt cognitive dysfunction, the presence of Veillonellaceae in a stool sample indicates that the subject has covert cognitive dysfunction and the presence of Lachnospiraceae indicates that the subject does not have covert cognitive dysfunction; and the presence of Streptococcaceae and/or Coriobacteriaceae in a saliva sample indicates that the subject has covert cognitive dysfunction. For subjects with a previous diagnosis of overt cognitive dysfunction, these tests are valuable because the subject is likely to have been treated, or to be receiving a treatment, and the methods disclosed herein enable practitioners to follow the progress of treatment. For example, a reversal of symptoms of overt cognitive dysfunction to covert cognitive dysfunction upon treatment would indicate that the treatment is helpful. Alternatively, if a patient previously treated for overt cognitive dysfunction regains normal cognition upon treatment, it would be helpful to know whether or not normal cognition is being maintained, or to know at an early stage if the patient is reverting to cognition problems, before overt symptoms appear.

In addition, the presence of specific microbes in stool samples were shown to correlate with a diagnosis of cognitive impairment in subjects suffering from cirrhosis and PTSD, these diagnoses being confirmed using standard known test methods. For such patients, the presence of at least one of Enterococcus, Proteobacteria, Ruminococcaceae and Lachnospiraceae is detected, and the presence of Enterococcus and/or Proteobacteria in the stool sample indicates that the subject has cognitive impairment, while the presence of Lachnospiraceae (if the subject has not had prior OHE) and the presence of Ruminococcaceae and Lachnospiraceae (if the subject has had prior OHE) indicates that the subject does not have cognitive impairment.

### Definitions

By “microbial signature” we mean the microbes that make up the microbial community that is present in a particular habitat, location or sample, e.g. the microbes that are present at a particular location in a host, such as in the gut, in saliva, on the skin, etc. Microbial signatures are known to differ from host to host, and some are known to be associated with (and possibly causally related to or symptomatic or indicative of) a particular disease, and/or with the absence of disease, i.e. some microbial signatures are associated with good or normal health.

Hepatic encephalopathy encompasses a broad range of neuro-psychiatric disturbances that may accompany portosystemic shunting, acute liver failure, and cirrhosis. Cirrhotic encephalopathy is broadly classified as overt and “minimal” or “convert” hepatic encephalopathy (MHE or CHE). MHE/CHE refers to the condition of that subset of patients with cirrhosis who do not have any clinically detectable neurologic abnormality but have abnormal neuropsychometric or neurophysiologic test results.

Posttraumatic stress disorder (PTSD) is a psychiatric disorder that can occur in people who have experienced or witnessed a traumatic event such as a natural disaster, a serious accident, a terrorist act, war/combat, rape or other violent personal assault.

Internal transcribed spacer (ITS) is the spacer DNA situated between the small-subunit ribosomal RNA (rRNA) and large-subunit rRNA genes in the chromosome or the corresponding transcribed region in the polycistronic rRNA precursor transcript.

The metabolome refers to the complete set of small-molecule chemicals found within a biological sample.

Metagenomics generally refers to the study of genetic material recovered directly from environmental samples. The broad field may also be referred to as environmental genomics, ecogenomics or community genomics. In contrast to methods which rely upon cultivated clonal cultures, metagenomics relies on e.g. direct gene sequencing of specific genes (often 16S rRNA gene) in a natural sample to produce a profile of diversity. The results are advantageously more accurate than relying on clonal cultures since, e.g. microbes that cannot be cultured in vitro can still be detected.

“Patient” or “subject” refers to mammals and includes human and veterinary subjects.

### Diagnosis

In some aspects, methods of diagnosing or identifying subjects with cognitive impairment (dysfunction). In some aspects, the cognitive impairment is covert cognitive dysfunction. The methods involve, for example, obtaining (e.g. collecting) a biological sample from the subject, typically a stool or saliva sample, and determining (measuring, quantifying, etc.) the amount (quantity, level) of one or more, usually all, of the microbes that are indicative of one or both of normal cognition and covert cognitive impairment. Those of skill in the art are familiar with methods of obtaining stool and saliva samples, e.g. via the use of swabs, deposition of sample directly into a receptacle (e.g. a tube), etc. It is noted that biological samples may be examiner or pre-tested for suitability for sequencing prior to use, e.g. stool samples obtained with swabs comprising lubricant were not acceptable.

In some aspects, the subject is already suffering from at least one chronic disease, e.g. a chronic disease that predisposes the subject to cognitive impairment. Examples of chronic diseases or conditions that predispose subjects to covert cognitive impairment include but are not limited to: liver diseases such as cirrhosis and chronic renal failure, alcohol misuse, non-alcoholic fatty liver, chronic liver disease, heart diseases such as heart failure, coronary artery disease; digestive diseases such as inflammatory bowel disease, irritable bowel syndrome, celiac disease, kidney disease such as chronic kidney disease, in patients with dialysis, psychiatric diseases such as post-traumatic stress disorder (PTSD), schizophrenia, bipolar disorder, anxiety, depression, autism, neurological diseases such as multiple sclerosis, ALS, Parkinson's disease, dementia; chronic obstructive pulmonary disease (COPD); morbid obesity; diabetes such as diabetes types I and II;

In other aspects, the subject may be known to be predisposed to cognitive impairment due to, e.g. age, overall health, and/or genetic factors.

In some aspects, the chronic disease/condition(s) is/are one or more of those listed by the Centers for Medicare and Medicaid Services, which include: alcohol abuse, drug abuse/substance abuse, Alzheimer's disease and related dementia, heart failure, arthritis (osteoarthritis and rheumatoid), hepatitis (chronic viral B & C), asthma, HIV/AIDS, atrial fibrillation, hyperlipidemia (high cholesterol), autism spectrum disorders, hypertension (high blood pressure), cancer (breast, colorectal, lung, and prostate), ischemic heart disease, chronic kidney disease, osteoporosis, chronic obstructive pulmonary disease (COPD), schizophrenia and other psychotic disorders, depression, stroke and diabetes. In some aspects, the chronic disease is hypertension; hyperlipidemia; arthritis; coronary artery disease; cancer; diabetes; or osteoporosis, or a combination of two or more of these.

The methods include a step of detecting the microbes (e.g. at least one microbe) in a biological sample from a subject suffering from a chronic disease. The detecting step allows characterization of the types of microbes that are present in a sample and establishment of a microbial signature. The microbiota can be characterized utilizing a broad range of molecular approaches that are useful for analyzing e.g. nucleic acid samples such as DNA and/or RNA, and/or proteins and/or products produced by microbes. While any number of suitable molecular techniques may be utilized, particularly useful molecular techniques include (i) screening of microbial 16S ribosomal RNAs (16s rRNA) using PCR and (ii) high-throughput “metagenome” sequencing methods, which detect over- and under-represented genes in the total bacterial population. Screening of 16s rRNA genes permits characterizing microorganisms present in the microbiota at the species, genus, family, order, class, and/or phylum level. Such screening can be performed, e.g., by conducting PCR using universal primers to the V2, V3, V4, V6 (or V2-V4) region of the 16s rRNA gene, followed by high-throughput sequencing and taxonomic analysis. See e.g., Gao et al. Proc. Natl. Acad. Sci. USA, 2007; 104:2927-32; Zoetendal et al., Mol. Microbiol., 2006, 59:1639-1650; Schloss and Handelsman, Microbiol. Mol. Biol. Rev., 2004, 68:686-691; Smit et al., Appl. Environ. Microbiol., 2001, 67:2284-2291; Harris and Hartley, J. Med. Microbial., 2003, 52:685-691; Saglani et al., Arch Dis Child, 2005, 90:70-73. The high-throughput “metagenome” sequencing methods involve obtaining multiple parallel short sequencing reads looking for under- and over-represented genes in a total mixed sample population. Such sequencing is usually followed by determining the G+C content or tetranucleotide content (Pride et al., Genome Res., 2003, 13; 145) of the genes to characterize the specific bacterial species in the sample. Additional techniques include those involving cultivation of individual microorganisms from mixed samples. See, e.g., Manual of Clinical Microbiology, 8th edition; American Society of Microbiology, Washington D.C., 2003.

In some aspects, the detection and characterization (identification) of microbes in a sample is carried out using e.g. a gene chip array of suitable probes e.g. oligomers specific for hybridization to nucleic acids of known bacteria, viruses, parasites and fungi. Targeted nucleic acids include but are not limited to: 18S rRNA gene, 5.8S rRNA gene, 28S rRNA gene, ITS1 and ITS2 for parasite detections, 16S rRNA gene for bacteria detections, 18S rRNA gene, ITS1, 5.8S rRNA gene, ITS2 and 26S rRNA gene to detect fungi, and conserved and specific viral genes to detect viral families and specific viruses.

In some aspects, the methodology is or includes a multiplex polynucleotide sequence analysis without the use of size separation methods or blotting, as described in issued U.S. Pat. No. 8,603,749, the complete contents of which is hereby incorporated by reference in entirety. Briefly, the multiplex method is used for determining abundance profiles of one or more target polynucleotide sequences across a plurality of samples. The steps of the method include amplifying and tagging target polynucleotides by PCR in each of a plurality of samples with an amplification primer comprising a high throughput sequencing adaptor, a sample-specific tag sequence of e.g. at least four nucleotides in length (e.g. a “bar code”), and a priming sequence to amplify targeted polynucleotide sequence(s) (e.g. 16S rRNA; combining polynucleotides amplified in the amplifying step to form a polynucleotide pool and sequencing the polynucleotide pool e.g. in high throughput. The step of sequencing to determine the sequence of a plurality (e.g. at least about 300) of tagged polynucleotides for each of the samples. In this aspect, the method also includes a step of assigning each nucleotide sequences to an originating sample by the nucleotide sequence of the sample-specific tag/bar code, thereby determining abundance profiles of the target polynucleotide sequence(s) across the samples.

Other techniques include global and deep molecular analysis e.g. using comprehensive, reproducible phylogenetic microarrays in combination with quantitative polymerase chain reaction; PCR amplification of 16S rRNA genes using primers specific for the 338-806 (V3-V4) hypervariable regions; sequencing; screening 16S rRNA genes using PCR in combination with high-throughput sequencing methods (e.g., pyrosequencing, etc.); real-time quantitative PCR, and/or high throughput sequencing; as well as Random Amplification of Polymorphic DNA PCR (RAPD-PCR); etc.

In particular, the methods that are used are or include the use of 16srRNA sequencing, quantitative PCR alone or in combination with other techniques, long-read single-molecule sequencing as applied directly to the overall amplified products generated in a PCR reaction (e.g. the MinION™ or similar technologies); various so-called next generation sequencing techniques (e.g. “next-gen” sequencing such as Ion Torrent™, 11lumina NextGen, etc.), and the like.

The data obtained by detecting microbes in a sample is processed to establish a microbial signature for the sample. Those of skill in the art are familiar with the analysis of such data, including statistical analyses to insure that the level of a microbe that is detected is significant. It is noted that microbiome data is non-parametric and non-parametric statistical techniques are used to evaluate such data. Techniques that may be used to establish or define a microbial signature, either as a reference or in a test sample, include but are not limited to: Machine Learning and SI deep learning; Ecological modeling; Complex Adaptive Systems modeling; Correlation network analysis; etc. Methods of establishing models of microbiome signatures are known in the art. See, for example, issued U.S. Pat. Nos.: 10,346,588; 10,364,474; 10,366,789; 10,380,325; and 10,383,519, the complete contents of each of which is herein incorporated by reference in entirely.

A diagnosis of cognitive dysfunction is made by comparing the microbial signature of the sample with one or more reference samples. Generally, when at least one of the types of microbes in a positive reference signature (based on individuals who have cognitive dysfunction) is detected, then the subject is diagnosed as having cognitive dysfunction. In addition, in this case, generally the microbes present in one or more negative controls (based on individuals who do not have cognitive dysfunction) are absent. Alternatively, when at least one of the types of microbes in a negative reference signature is detected, then the subject is diagnosed as not having cognitive dysfunction, and generally the microbes present in one or more positive controls are absent.

The results of a comparison may be “all or nothing”, e.g. a microbe is either present or not present (detectable or undetectable). Alternatively, and/or in addition, a microbe may be present but the level of the microbe may differ from that of one or more reference or control values, when assessed using the same type of sample (e.g. stool or saliva), e.g. the relative abundance of microbes of interest may be measured.

As discussed above, the amount and/or relative abundance is compared to one or more suitable control or reference values or levels, e.g. a reference microbiome for a particular sample type, such as stool or saliva. Those of skill in the art are aware of methods to develop control values. In some aspects, the control group that is used to develop a negative control values is a healthy population, e.g. subjects who do not have any cognitive dysfunction and also do not have a chronic disease. In addition, or alternatively, a negative control values may be developed based on subjects who have the chronic disease of the test subject, but do not have cognitive dysfunction. These first two scenarios are negative controls, and the results of a subject with cognitive dysfunction will differ from those of the negative control.

In other aspects, positive control values are established, e.g. values based on sampling from subjects known to have cognitive dysfunction. When positive reference values are used, when the types and levels of microbes that are detected in a test subject are the same (or similar enough to be statistically significant and considered as the same) as those of the positive reference, then the subject is considered to belong to the category of subjects who do have covert cognitive dysfunction. Generally, in this case, the microbes present in a negative control microbial signature are not detected, or are detected at statistically significant lower levels.

In some aspects, the subject who is tested has a chronic disease, such as cirrhosis, has not had a prior diagnosis of overt hepatic encephalitis, and the subject is tested for CHE. In such subjects, the presence of Veillonellaceae in a stool sample indicates that the subject has CHE and the presence of Lachnospiraceae indicates that the subject does not have CHE; and the presence of Lactobacillaceae and/or Coriobacteriaceae in a saliva sample indicates that the subject has CHE; and the presence of Clostridiales clusterXI, Aerococcaceae and/or Prevotellaceae indicates that the subject does not have CHE.

In other aspects, the methods are used in subjects who have a chronic disease or condition that predisposes them to cognitive dysfunction, but who have not yet progressed even to covert cognitive dysfunction, as measured by other test methods (e.g. Stroop, etc). The subjects may have been deemed to be free of CHE. In such subjects, the results of the analysis may be intermediate between those of positive and negative controls, indicating that the subject is in the process of developing CHE, and one or more treatment measures disclosed herein can be undertaken to prevent progression to covert cognitive dysfunction. This is especially advantageous as these subjects would be overlooked for treatment, without the tests provided herein. In addition, some subjects who are negative for cognitive dysfunction by standard tests may also be negative using the methods described herein, but can benefit from receiving a treatment prophylactically. In such cases, the dose of e.g. a drug, may be lower than that used for full-blown cognitive dysfunction, and/or drugs may be advantageously avoided altogether in favor of life-style changes.

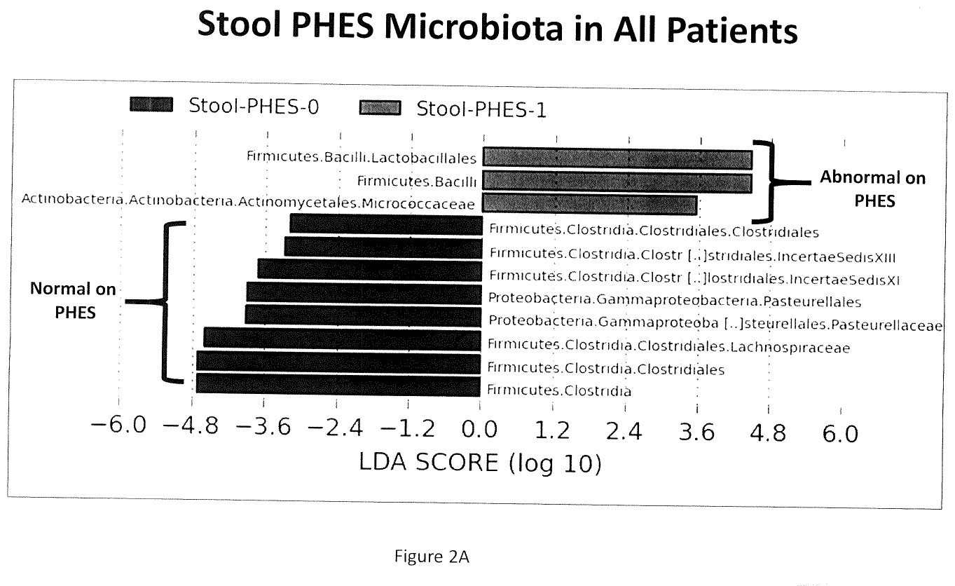

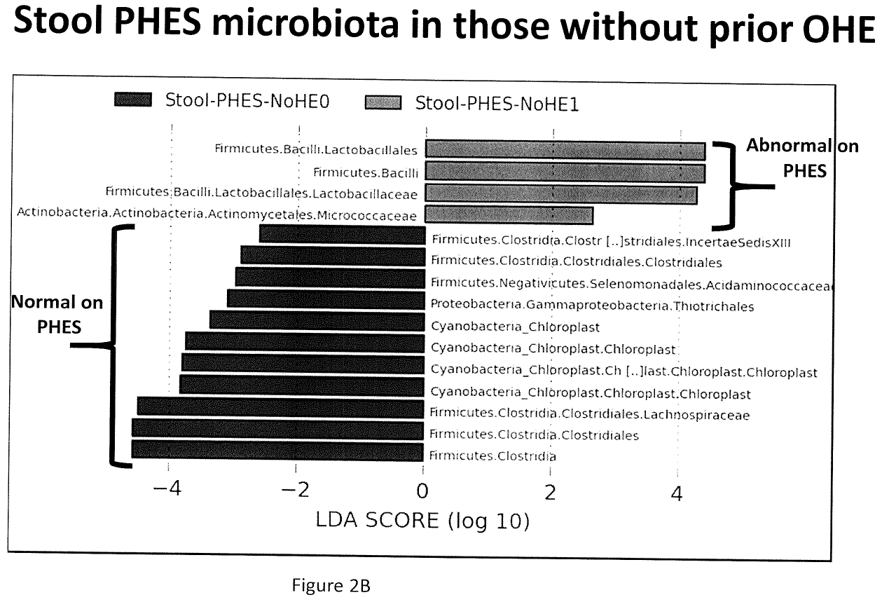

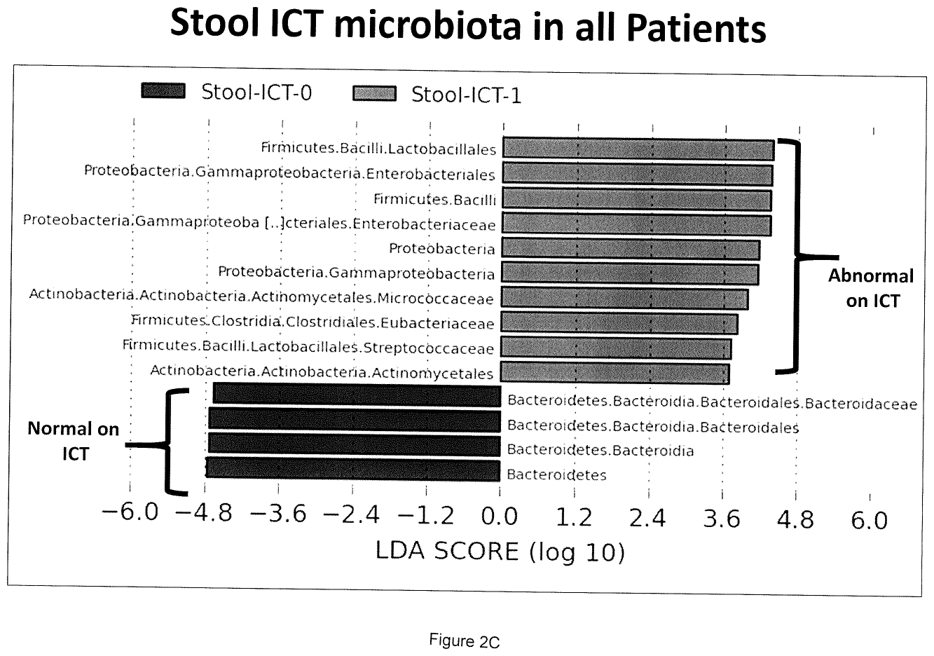

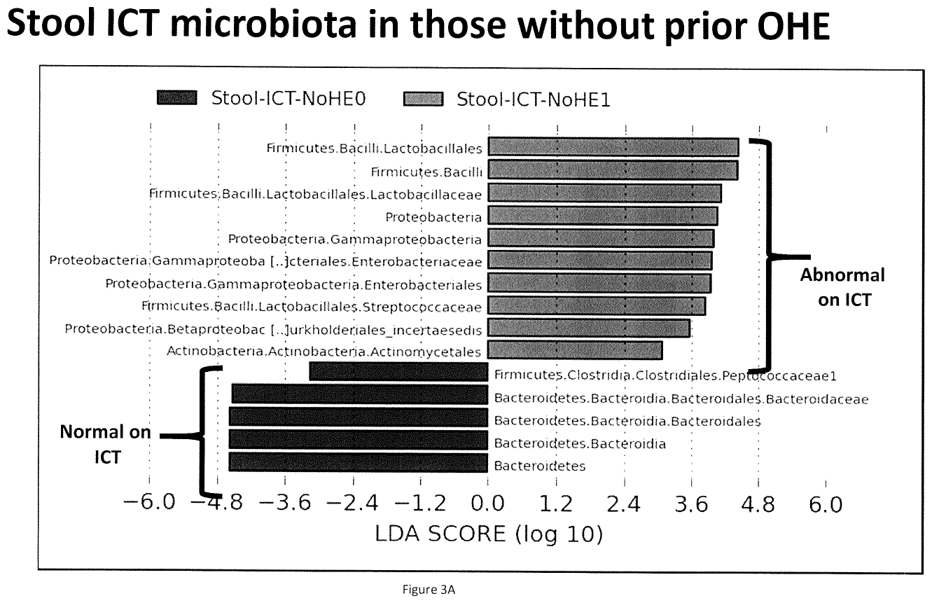

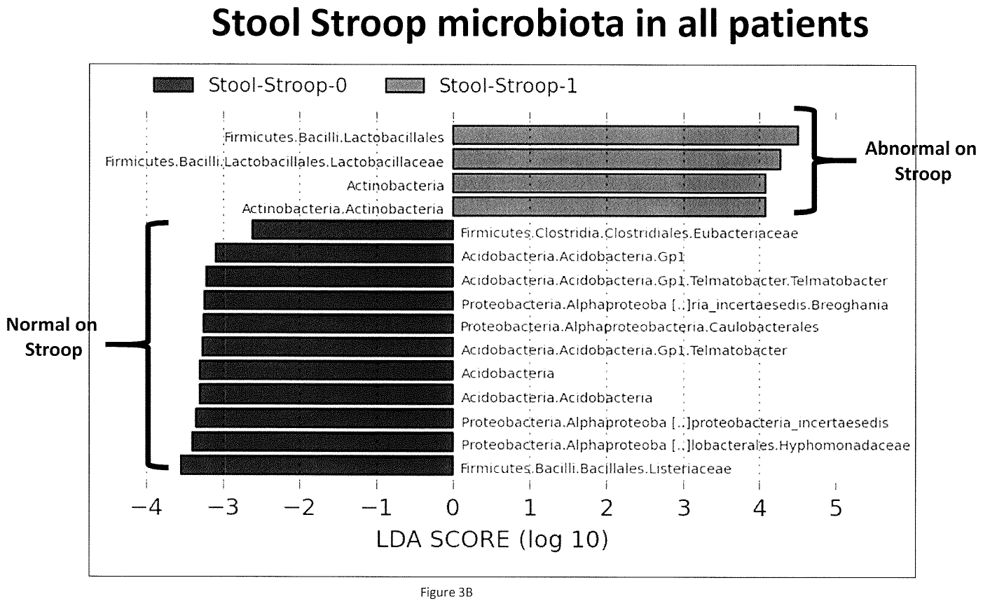

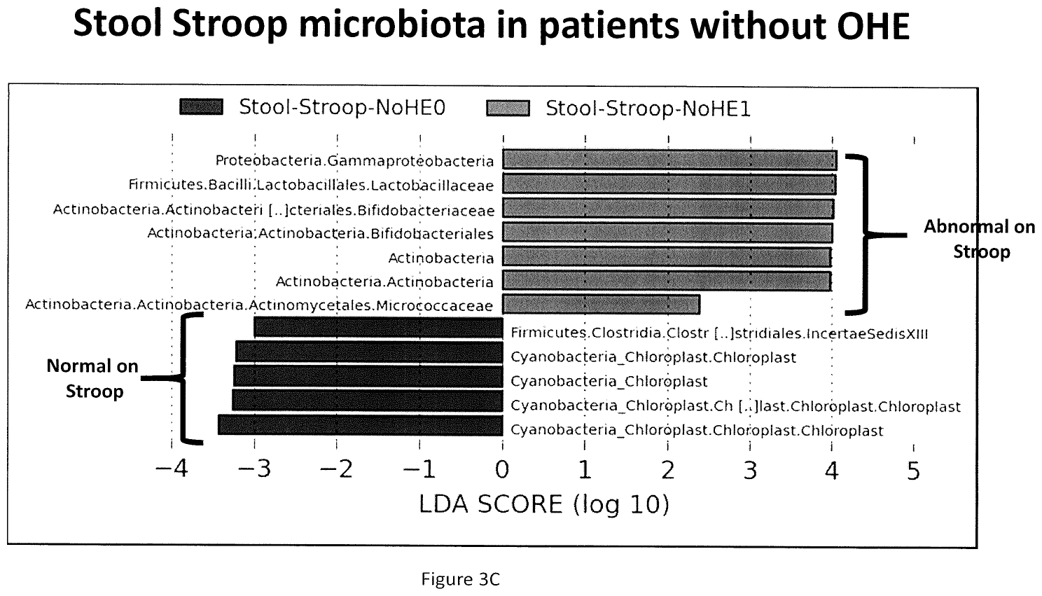

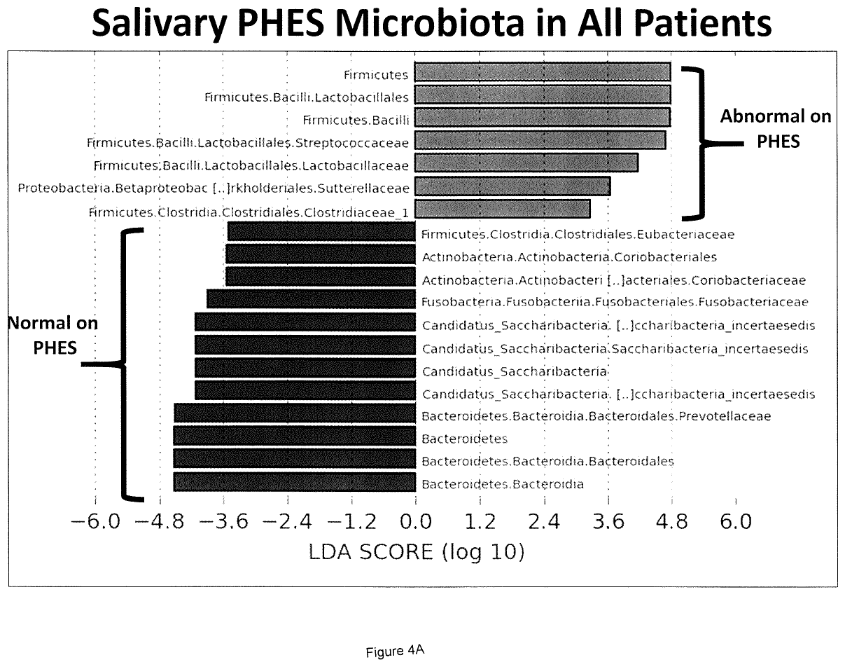

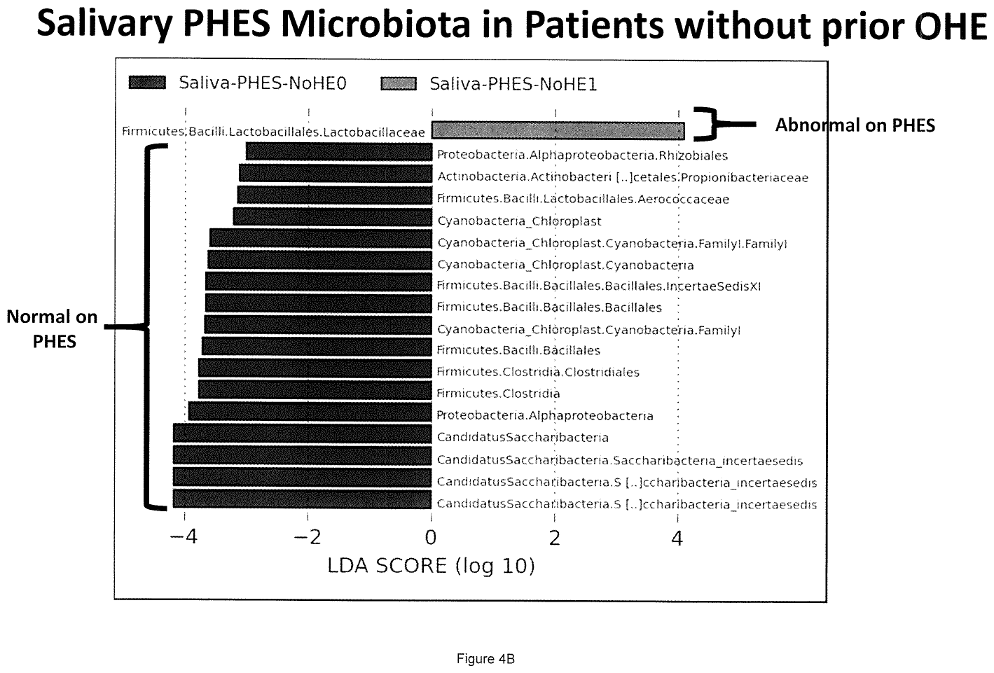

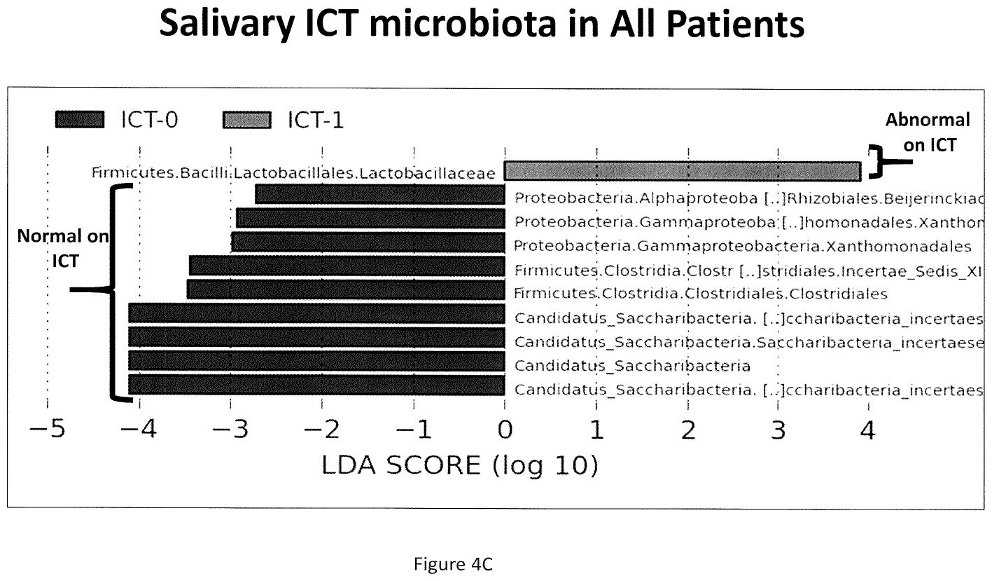

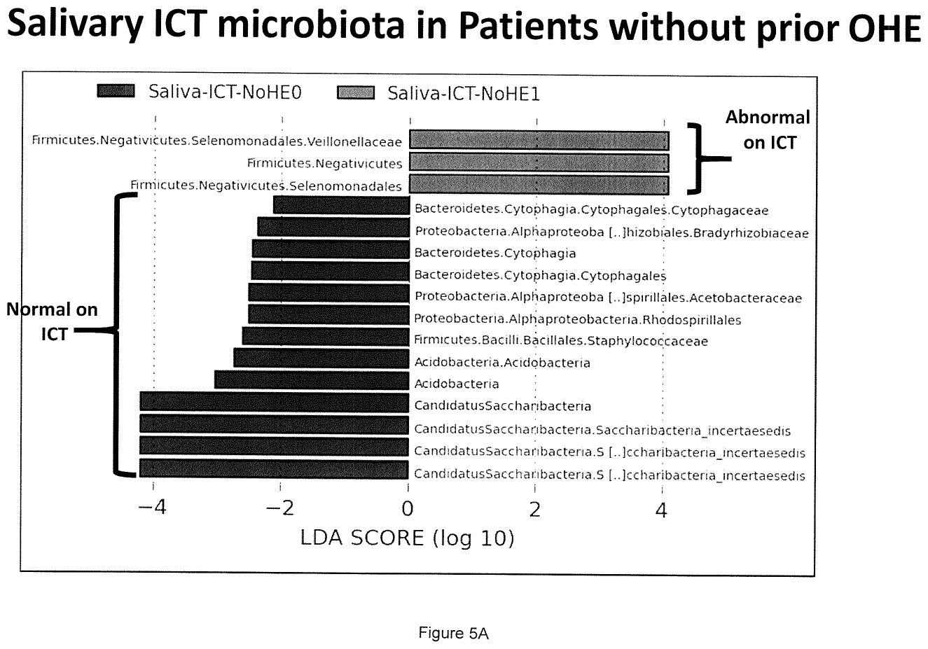

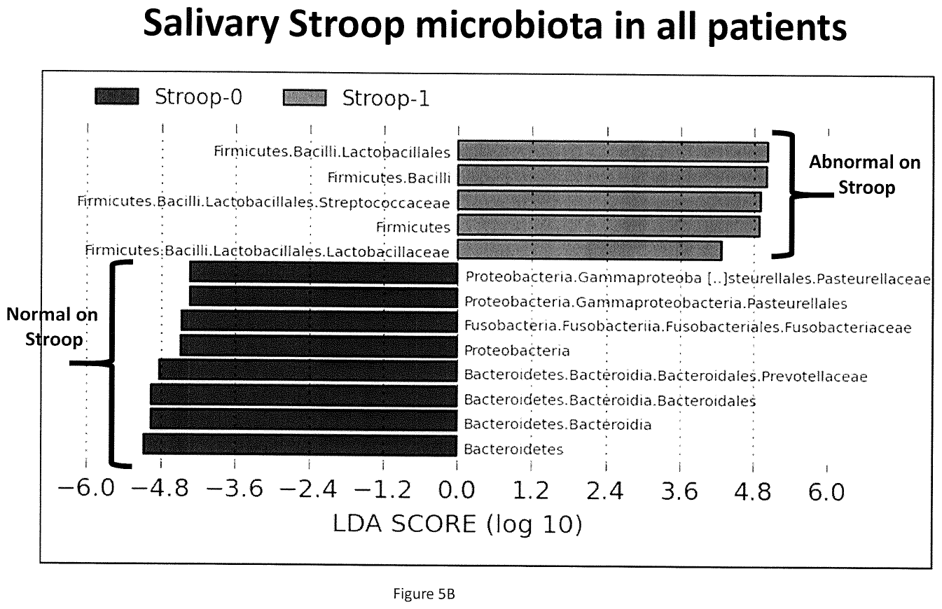

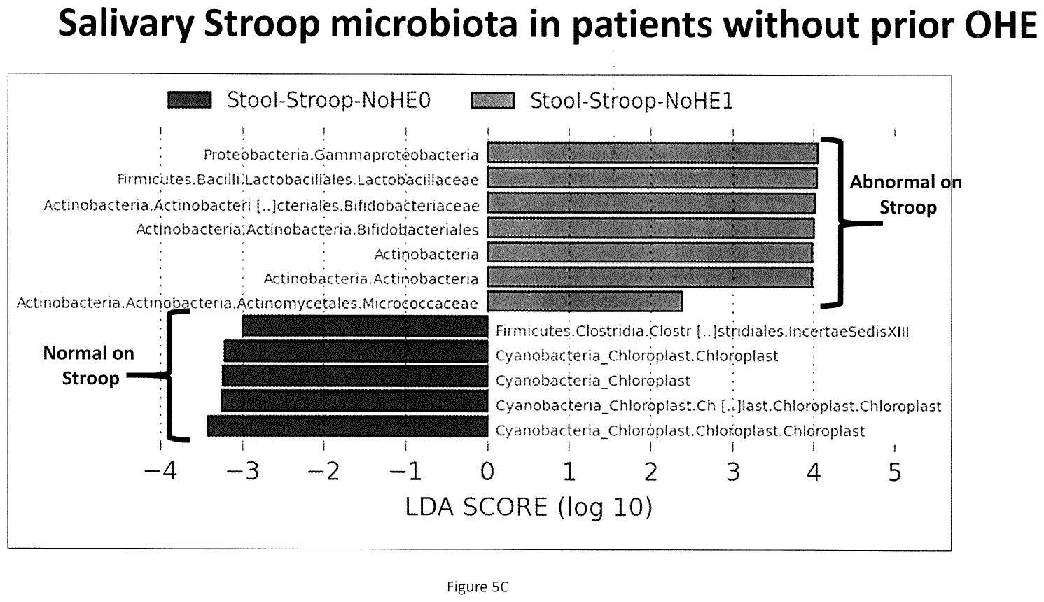

In other aspects, other microbes may be detected, including but not limited to fungi, archeaea and viruses that are present in the gut (e.g. stool), in saliva and on other surfaces. Exemplary additional microbiota that may be detected are shown in FIGS. 2-5 and include but are not limited to one or more of the following phyla, orders, classes, families and genera, etc.:

in stool: Actinobacteria, Actinomycetales, Micrococcaceae, Bacilli, Firmacutes, Clostridia, Proteobacteria, Pasteurellaceae, Gammaproteobacteria,InsertaeSedis XI, InsertaeSedis XIII, Negativicutes, Selenomonadales, Acidiminococcaceae, Thiotricales, Cyanobacteria (chloroplast), Enterobacteriales, Enterobacteriaceae, Eubacteriaceae, Streptococcaceae, Bacteroidetes, Negativicutes, Selenomonadales, Acidiminococcaceae, Thiotricales, Cyanobacteria (chloroplast), Enterobacteriales, Enterobacteriaceae, Peptococcaceae 1, Acidobacteria, Acidobacteria Gp1, Telmatobacter, Alphaproteobacteria, Incertaesedis, Breoghania, Caulobacterales, Hyphomondaceae, Listeriaceae, Bifidiobacteriaceae andBifidobacteriales;

in saliva: Firmacutes, Bacilli, Lactobacilliales, Lactobacilliaceae, Proteobacteria, Gammaproteobacteriales, Enterobacteriaceae, Actinobacteria, Actinomycetales, Micrococcaceae, Clostridia, Clostridiales, Eubacteriaceae, Streptococcaceae, Bacteroidetes, Bacteroidales, Bacteroidaceae, Alphaproteobacteria, Rhizobiales, Propionibacteriaceae, Aerococcaceae, Cyanobacteria (chloroplast), Cyanobacteria, Cyanobacteria familyl, Candidatus Saccharibacteria, Saccharibacteria_incertaesedis, Beijerinckiaceae and Xanthomondales.

In some aspects, the subject has a chronic disease, such as cirrhosis, and also has a second diagnosis. In some aspects, the second diagnosis is PTSD. In such subjects, the microbial signature is established by, in a stool sample, detecting the presence of at least one of Enterococcus, Proteobacteria, Ruminococcaceae and Lachnospiraceae. The presence of Enterococcus and/or Proteobacteria in the stool sample indicates that the subject has cognitive impairment, and the presence of Ruminococcaceae and/or Lachnospiraceae indicates that the subject does not have cognitive impairment. In particular, if the subject has not previously had OHE, then the presence of Lachnospiraceae indicates that the subject does not have cognitive impairment; and if the subject has previously had OHE, the presence of Ruminococcaceae and/or Lachnospiraceae indicates that the subject does not have cognitive impairment.

In other aspects, other microbes may be detected in subjects with PTDS, including but not limited to fungi, archeaea and viruses that are present in the gut (e.g. stool), in saliva and on other surfaces. Exemplary additional microbiota that may be detected are shown in Table 9 and include but are not limited to one or more of the following phyla, orders, classes, families and genera, etc.: in stool: Enterococcaceae_Pillibacter; Streptococcaceae_Streptococcus; Acidaminococcaceae_Acidaminococcus; Gracilibacteriaceae_Lutispora; Clostridiaceae_Proteiniclasticum; Thermodesulfobiaceae_Thermodesulfobium (present in subject with PTSD and a cognitive disorder) and Lachnospiraceae_Ruminococcus; Lachnospiraceae_Roseburia; Lachnospiraceae_Anaerostipes; Lachnospiraceae_ClostridiumXIVa; Lachnospiraceae_Eisenbergiella; Lachnospiraceae_Lachnospira; Ruminococcaceae_Pseudoflavonibacter; Ruminococcaceae_Subdoligranulum, (present in subjects with no PTSD and no cognitive disorder).

### Treatment

In some aspects, the methods include a step of treating a subject identified as having cognitive dysfunction, such as a covert cognitive dysfunction. In some aspects, the covert cognitive dysfunction is CHE. The methods may include a step of determining the microbiotic signature of a sample from the subject as described herein, and if the subject is identified as having a microbiotic signature corresponding to a diagnosis of covert cognitive dysfunction as described above, then providing one or more suitable treatments to the subject.

The treatments that are undertaken reverse, slow (delay) the onset or progression of, or lessen the severity of, at least one symptom of cognitive dysfunction, and/or prevent, slow (delay) the onset or progression of, or lessen the severity of at least one symptom of cognitive dysfunction. For example, the treatment methods may prevent the progression to CHE, or the progression of CHE to OHE, and may in fact reverse symptoms of CHE.

Symptoms of CHE include but are not limited to: difficulty with cognitive abilities such as executive decision-making and psychomotor speed; abnormalities in psychometric testing and slow response time; etc. In CHE, there are no clinical signs or symptoms of OHE; however, patients have neuropsychological deficiencies that can be detected with psychometric or neuropsychological testing. Unfortunately, patients with CHE tend to have poor quality of life, diminished work productivity, and increased traffic violations and accidents. Any of these symptoms can be avoided or reversed by the methods described herein. For example, one or more of executive decision-making ability, psychomotor speed and performance in psychometric testing can be avoided or improved and in some cases, a subject with CHE can revert to normal performance.

In OHE, there is a wide spectrum of symptomatic presentation and using the methods described herein one or more of the following symptoms can be avoided: grade symptoms include increased fatigue, poor short-term memory and concentration, and insomnia; grade 2 symptoms include confusion, changes in personality, bizarre behavior, and slurred speech; grade 3symptoms include stupor; and grade 4 is characterized by coma, either responsive or unresponsive to noxious stimuli.

In some aspects, the treatment is or includes administration of one or more of rifaximin, lactulose, probiotics, prebiotics, synbiotics, postbiotics, organ transplants, calcineurin inhibitors (CNIs) such as cyclosporine and tacrolimus, fecal transplants, administration of fecal matter from healthy donors, treatment for hepatitis B and hepatitis C, treatments for diabetes, hypertension and other related medical conditions, detoxification programs, and immunological therapies.

In some aspects, the cognitive dysfunction is CHE and the treatment includes administering: an antibiotic such as rifaximin, neomycin, metronidazole, etc. e.g. to reduce ammonia levels in the blood, for example, by decreasing ammonia-producing colonic bacteria; or administering another agent that reduces ammonia levels such as lactulose, lactilol, lactose; or an agent that increases ammonia metabolism, e.g. ornithine aspartate, sodium benzoate, phenylacetate, etc. In some aspects, the treatment methods include one or more types of bacteriotherapy. “Bacteriotherapy” refers to the use of probiotics; fecal matter transplants (FMT)/intestinal microbiota transplant (IMT), especially from a so-called “super donor” whose fecal matter is highly diverse; and synbiotics which combine prebiotics (indigestible ingredients that promote growth of beneficial microorganisms) and probiotics. Post-biotics or non-living products of microbiota, specific phages and phage cocktails are also used.

Examples of probiotics and synbiotics that have been demonstrated to be effective in treating, for example, CHE, include but are not limited to:
administration of Lactobacillus acidophilus, L. rhamnosus, Bifidobacterium longum and Saccharomyces boulardi (Saji S, Kumar S, Thomas V. Trop Gastroenterol 2011 April-June; 32(2): 128-32 ;
administration of L-ornithine L-aspartate (Butterworth et al. J Clin Exp Hepatol. 2018 September; 8(3):301-313);
administering Streptococcus faecalis, Clostridium butyricum, Bacillus mesentricus (Sharma P, Sharma BC, Puri V, Sarin SK. An open-label randomized controlled trial of lactulose and probiotics in the treatment of minimal hepatic encephalopathy. Eur J Gastroenterol Hepatol 2008 June; 20(6):506-11;
administering probiotic yogurt (Bajaj J S, Saeian K, Christensen K M, et al. Probiotic yogurt for the treatment of minimal hepatic encephalopathy. Am J Gastroenterol 2008 July; 103(7):1707-15);
administering a synbiotic preparation (Liu Q, Duan Z P, Ha D K, Bengmark S, Kurtovic J, Riordan S M. Synbiotic modulation of gut flora: effect on minimal hepatic encephalopathy in patients with cirrhosis. Hepatology 2004; 39(5):1441-9;
administering Bifidobacterium longum with fructo-oligosaccharide (Malaguarnera M, Greco F, Barone G, Gargante M P, Malaguarnera M, Toscano M A. Bifidobacterium longum with fructo-oligosaccharide (FOS) treatment in minimal hepatic encephalopathy: a randomized, double-blind, placebo-controlled study. Dig Dis Sci 2007 November; 52(11):3259-65.; etc.

In some aspects, the bacteriotherapy involves administering one or more microbes that are shown to be missing from the microbial signature of a subject diagnosed with cognitive dysfunction. For example, the bacteriotherapy may include administration of one or more of Lachnospiraceae, Clostridiales clusterXI, Aerococcaceae, Prevotellaceae and Ruminococcaceae. In particular, in all patients tested, the presence of Lachnospiraceae was associated with a healthy microbiome and a lack of cognitive dysfunction, and its administration is preferred.

In other aspects, and/or in addition, life-style changes are prescribed, generally in combination with a medication. Such changes include but are not limited to: increased exercise; weight loss; consuming a brain-supportive diet (such as consumption of oily fish; omega 3 fatty acid supplements; cacao; berries; nuts; seeds; fruits; whole grains; coffee; tea; avocados; peanuts; leafy green vegetables; beans; lentils; herbs and spices with high antioxidant levels; vegetables high in fiber and inulin; fermented foods such as kimchi, sauerkraut, yogurts, kefir; and avoiding red meat, butter, high-fat dairy products and alcohol); increasing social connections; learning new tasks, etc.

### Monitoring

In some aspects, the methods disclosed herein are also used to monitor the progress of treatment, e.g. with one or more or the modalities described below, and/or to monitor the progress of cognitive abilities, onset of dysfunction, etc. in a subject with or without treatment.

For example, a patient who is known to be susceptible to cognitive dysfunction, e.g. a subject with a chronic disease or condition as described above, but who has normal cognition and has not been previously diagnosed with cognitive dysfunction, can be monitored non-invasively using the present methods, and treated at an early stage if cognitive decline is detected. Alternatively, a subject already diagnosed with a cognitive disorder (either covert or overt) using either the present methods or other criteria, can be monitored while being treated. If cognitive function improves during treatment, the treatment may be continued. However, if cognitive function remains stable or declines, a skilled practitioner may change the treatment method, e.g. by increasing the dosage of a drug, or adding one or more additional treatment measures, etc.

### Data Processing

In some aspects, a first method of developing control or reference models includes: receiving an aggregate set of biological samples from a population of subjects; characterizing a microbiome composition (taxa, genera, species, strains and other levels of taxonomy) and functional features (genes/and or metabolome, e.g. products such as metabolites, RNA and proteins) for each of the aggregate set of biological samples associated with the population of subjects, thereby generating at least one of a microbiome composition dataset and a microbiome functional dataset for the population of subjects; receiving a supplementary dataset, associated with at least a subset of the population of subjects, wherein the supplementary dataset is informative of characteristics associated with a cognitive dysfunction; and generating a characterization of the cognitive dysfunction based upon the supplementary dataset and features extracted from at least one of the microbiome composition dataset and the microbiome functional dataset. In some variations, the method can further include: based upon the characterization, generating a therapy model configured to correct the cognitive dysfunction.

In some aspects, the method functions to generate reference, control or characterization models that can be used to characterize and/or diagnose individual test subjects according to at least one of their microbiome composition and functional features (e.g., as a clinical diagnostic, as a companion diagnostic, etc.), e.g. by comparison to a reference/characterization model, and provide therapeutic measures (e.g., probiotic-based therapeutic measures, drug-based therapeutic measures, clinical measures, etc.) indicated by a therapy model to subjects based upon microbiome analysis for a population of subjects. As such, data from the population of subjects can be used to characterize subjects according to their microbiome composition and/or functional features, indicate states of health and areas of improvement based upon the characterization(s), and promote one or more therapies that can modulate the composition of a subject's microbiome toward one or more of a set of desired equilibrium states. Generally, the equilibrium states are the same or similar to those of normal control populations. Variations of the method can further facilitate monitoring and/or adjusting of therapies provided to a subject, for instance, through reception, processing, and analysis of additional samples from a subject throughout the course of therapy. In specific examples, the method can be used to promote targeted therapies to subjects suffering from cognitive dysfunction. In specific examples, the method can be used for characterization of and/or therapeutic intervention for one or more of: CHE, OHE, etc.

As such, in some embodiments, one or more outputs of the first method can be used in a second method to generate diagnostics and/or provide therapeutic measures for an individual test subject based upon an analysis of the test subject's microbiome composition and/or functional features of the subject's microbiome. Functional features include, for example, products (metabolites) produced by microbes or as a result of the presence of microbes, disease symptoms known to be caused by the presence of particular microbes, etc. In this aspect, the second method can include: receiving a biological sample from a subject; characterizing the subject as having a cognitive dysfunction based upon processing a microbiome dataset derived from the biological sample; and promoting a therapy to the subject with the immune microbial dysfunction based upon the reference model(s) and the therapy model(s) generated by the first method.

The methods thus function to generate models that can be used to classify individuals and/or provide therapeutic measures (e.g., therapy recommendations, therapies, therapy regimens, etc.) to those individuals, based upon prior microbiome and/or functional analysis for a population of individuals. As such, individuals are classified according to their microbiome compositions (e.g., as a diagnostic measure), to indicate states of health and areas of improvement based upon the classification(s), and/or provide therapeutic measures that can push the composition of an individual's microbiome toward one or more of a set of improved equilibrium (e.g. normal, non-disease) states. Variations of the second method can further facilitate monitoring and/or adjusting therapies provided to an individual, for instance, through reception, processing, and analysis of additional samples from an individual throughout the course of therapy.

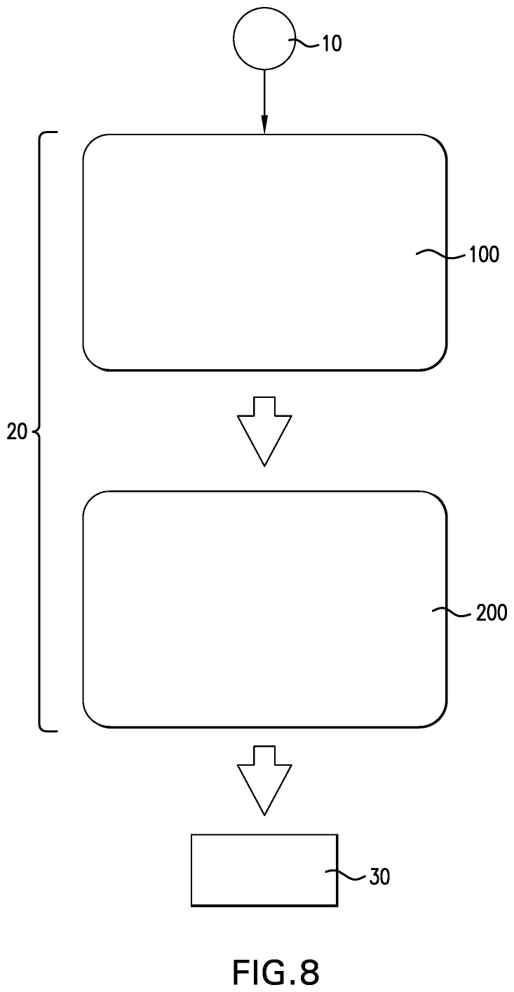

In some aspects, at least one of the methods is implemented, at least in part, at a system 20, as shown in the schema in FIG. 8 and described below, that receives a biological sample derived from the subject (or an environment associated with the subject) by way of a sample collection kit, and processes the biological sample at a processing system implementing a characterization model configured to classify the subject and a therapy model configured to recommend a course of treatment to positively influence a microorganism distribution in the subject (e.g., human, non-human animal, environmental ecosystem, etc.). In variations of the application, the processing system can be configured to generate and/or improve and/or refine the characterization process and the therapy model based upon sample data received from an additional population of subjects. The methods can, however, alternatively be implemented using any other suitable system(s) configured to receive and process microbiome-related data of subjects, in aggregation with other information, in order to generate models for microbiome-derived diagnostics and associated therapeutics. These can include microarrays, Quantitative (real-time) PCR, (qPCR), RNA sequencing and other methods. Thus, the methods can be implemented for a population of subjects (e.g., including the subject, excluding the subject), wherein the population of subjects can include patients dissimilar to and/or similar to the subject (e.g., in health condition, in dietary needs, in demographic features, etc.). Thus, information derived from the population of subjects can be used to provide additional insight into connections between behaviors of a subject and effects on the subject's microbiome, due to aggregation of data from a population of subjects.

Accordingly, the methods can be implemented for a population of subjects (e.g., including the subject, excluding the subject), wherein the population of subjects can include subjects dissimilar to and/or similar to the subject (e.g., health condition, in dietary needs, in demographic features, etc.); and information derived from the population of subjects can be used to provide additional insight into connections between behaviors of a subject and effects on the subject's microbiome, due to aggregation of data from a population of subjects.

In some aspects, with reference to FIG. 8, the methods are implemented in a system 20 comprising sample processing unit 100 for receiving sample 10 (e.g. a stool or saliva sample, or portion thereof) obtained, for example, from a collection kit. In sample processing unit 100, the sample is processed e.g. by contacting the sample with a substrate (such as a gene chip) comprising immobilized nucleic acid probes specific for binding to e.g. 16S rRNA of a plurality of microbes that have been identified as associated with one or both of i) a population of negative control individuals who do not have a cognitive dysfunction and ii) a population of positive control individuals who have a cognitive dysfunction. The step of contacting is performed under conditions that permit fragmenting nucleic acid material in the sample; hybridizing a primer present on the substrate, and previously identified as specific for a nucleic acid sequence, e.g. 16S rRNA, of a microbe associated with a cognitive dysfunction, determining a microorganism nucleic acid sequence by amplifying the fragmented nucleic acid material based on the identified primer; and determining an alignment of the microorganism nucleic acid sequence to a reference nucleic acid sequence associated with the cognitive dysfunction; generating a microbiome composition dataset and a microbiome functional dataset for the population of subjects based upon the alignments; collecting a supplementary dataset, associated with at least a subset of the population of subjects, wherein the supplementary dataset is informative of a characteristic associated with the IBD condition; generating a characterization of the IBD condition based upon the supplementary dataset and at least one of the microbiome composition dataset and the microbiome functional diversity dataset; based upon the characterization, generating a therapy model that determines a therapy for correcting the IBD condition; and at an output device associated with the subject, providing the therapy to the subject with the IBD condition based upon the characterization and the therapy model, wherein the therapy modulates microbiome composition of the subject towards an equilibrium state.

Data regarding which microbes are detected in the sample is passed to a computerized processing unit 200, where the data is compared to previously established characterization models, and optionally a therapy model. Output 30 from processing unit 200 includes one or more of a classification of the subject as having or not having cognitive dysfunction, and a recommended treatment regimen for treating, preventing, reducing, etc. at least one symptom of the cognitive dysfunction. In some aspects, the treatment causes the gut microbiome of the treated subject to return to a microbiome composition known to be the same or similar to a microbiome characteristic of normal, non-diseased subjects. In other aspects, a change in the microbiome is not possible or does not occur, but one or more symptoms of the cognitive dysfunction can still be alleviated, lessened, eradicated, etc. by the treatment.

Further details of sample collection, sample processing and data processing are found, for example, in issued U.S. Pat. No. 10,346,588 (incorporated by reference in entirety elsewhere herein).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### EXAMPLES

### Example 1

Specific Gut and Salivary Microbiota Patterns Are Linked with Different Cognitive Testing Strategies in Covert Hepatic Encephalopathy (Covert and Minimal Hepatic Encephalopathy are used inter-changeably)

### Methods:

Outpatients with cirrhosis were enrolled from hepatology clinics at the Virginia Commonwealth University and Richmond VA Medical Center after written informed consent. Patients were diagnosed with cirrhosis based on any of the following, liver biopsy, transient elastography, evidence of varices, nodular contour of liver or thrombocytopenia in a patient with chronic liver disease or frank decompensation of cirrhosis. Patients with an unclear cirrhosis history, those unable to provide consent, patients with current alcohol or illegal drug abuse, those on anti-psychotic, anti-seizure, older anti-depressants or benzodiazepine usage, those with recent TIPS (<3 months), with recent changes in opioid medications (over the last 3 months) and those with recent (<1 month) hospitalizations were excluded. Patients on stable SSRI or SNRI anti-depressants and those on stable doses of opioid therapy (for >3 months) were included.

Every patient was administered the mini-mental status exam (MMSE) and only those with a score of ≥25 were given the specialized cognitive tests. Patients underwent testing with any of the following validated strategies for CHE (a) Psychometric hepatic encephalopathy score (PHES) (14) (b) Inhibitory Control test (ICT) (15) and (c) EncephalApp Stroop (16) during the same sitting in this order. We administered PHES to everyone, while a subset also underwent ICT and EncephalApp Stroop testing. CHE was diagnosed on US-based norms (17).

### Cognitive Testing Details:

PHES consists of 5 tests, the standard deviations of which are compared against healthy controls and the total sum is added. A low total score against the reference control population indicates poor performance. EncephalApp Stroop has two sections, an easier Off state where the subject has to recognize the color of the # signs appropriately and touch the screen at the corresponding color, and a more difficult On state where the words meaning specific colors are presented in discordant colors. The time to complete 5 correct runs in each state are added with the total time, OffTime, OnTime and the number of runs required to complete 5 states. A higher time required indicates poor performance ICT is a computer-based in which subjects are shown a series of letters and are asked to respond by pressing a mouse key when an X is followed by a Y or a Y is followed by an X (alternating presentation, termed targets). Patients are instructed not to respond to X following X or Y following Y (non-alternating presentation, termed lures). High lure and low target response indicate poor psychometric performance. The ICT is administered as a practice test followed by a series of 6 similar 2- minute runs, separated by breaks to allow the subjects to rest. There are total of 212 targets and 40 lures scattered throughout the test. Weighted lures are lures divided by the square of target accuracy/100 (18).

### Microbiota Analysis:

In addition, patients provided a stool sample and a subset also provided salivary samples using published techniques on the same day (19). 16SrRNA microbiota analysis was performed using Multitag™ sequencing on an Ion Torrent PGM as previously published (20). The main objectives were to determine the microbial taxa that differentiated between patients who had CHE on individual modalities compared to the rest in the entire group and the subset without prior OHE. All analyses were performed separately for stool and salivary microbiota. We used LEFSe (Linear Discriminant Analysis Effect Size) to determine the taxa that differentiated the groups (21).

Ultimately, these taxa were then introduced into a logistic regression model with clinical variables of defining cognitive impairment. Variables significant at p<0.20 on univariate analysis were introduced into the final models and backward logistic regression models were used to predict specific cognitive impairments. The clinical variables used were age, gender, education, prior OHE (for the entire cirrhosis group), proton pump inhibitor (PPI) use and MELD score. In the no-OHE group, all the above clinical variables were input apart from prior OHE. Microbiota families were input on phylum at a time for data reduction and specific families that were p<0.20 were input with the clinical variables for the final model.

For families that emerged significant in the prediction of the logistic regression models, we further analyzed them at the genus level to determine the specific genera associated with specific cognitive impairments or with normal cognitive function.

### Results:

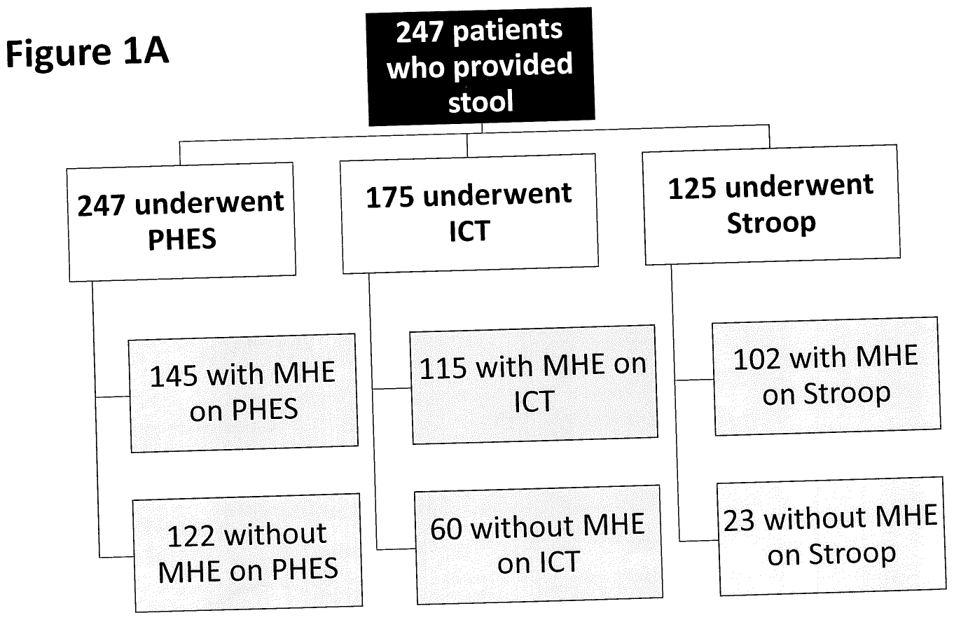

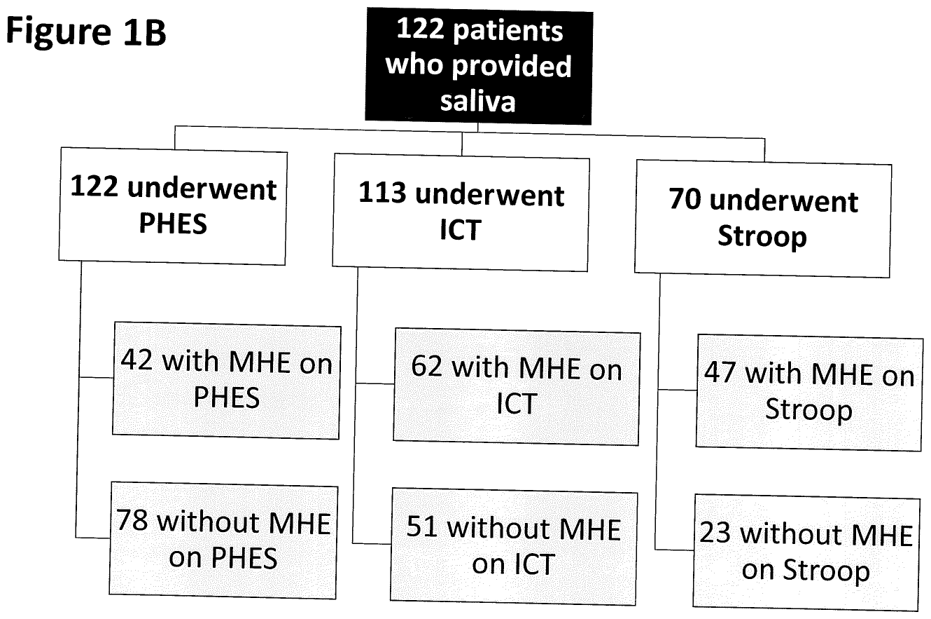

267 patients with cirrhosis were enrolled, all of whom underwent at least PHES testing and stool collection. Of these patients, 123 had prior HE (118 on lactulose and 77 on rifaximin). 175 patients underwent additional ICT and 125 were also given EncephalApp Stroop tests. A subset of patients (n=112) also gave saliva. The details of CHE diagnosis using the individual modalities in patients who underwent stool and saliva collection are shown in FIGS. 1A and B. As shown in Table 1, patients with OHE were more likely to be men, be on PPI, have a higher MELD score and worse cognitive performance than patients without prior OHE. Most patients with prior OHE were on lactulose and rifaximin and none of the patients without prior OHE were on any of these medications. Details of patients who gave saliva are shown in Table 2.

TABLE 1
Details of patients with stool collection and PHES scoring

| | No prior Overt | Prior Overt HE | |
|---|---|---|---|
| | HE (n = 144) | (n = 123) | P value |
| Age (years) | 59.2 ± 7.1 | 59.1 ± 7.3 | 0.91 |
| Education (years) | 13.7 ± 2.2 | 13.4 ± 2.3 | 0.26 |
| Gender (male) | 96 (67%) | 110 (89%) | <0.0001 |
| Proton Pump Inhibitor Use | 58 (41%) | 74 (60%) | 0.001 |
| Alcoholic etiology of cirrhosis | 39 (27%) | 30 (24%) | 0.42 |
| MELD score | 10.7 ± 5.5 | 15.6 ± 6.1 | <0.0001 |
| Lactulose | — | 118 | — |
| Rifaximin | — | 77 | — |
| CHE (yes/no) | | | |
| PHES (n = 267 total) | 63 (44%) | 82 (67%) | <0.0001 |
| ICT (n = 175 total) | 54 of 87 (62%) | 61 of 88 (69%) | 0.31 |
| Stroop (n = 125 total) | 35 of 49 (71%) | 67 of 76 (88%) | 0.02 |
| PHES components | | | |
| Number connection A (seconds) | 38.0 ± 17.4 | 57.6 ± 36.0 | <0.0001 |
| Number connection B (seconds) | 97.3 ± 48.6 | 174.0 ± 127.0 | <0.0001 |
| Digit symbol (seconds) | 53.4 ± 17.6 | 39.2 ± 14.9 | <0.0001 |
| Line tracing time (seconds) | 101.0 ± 35.5 | 128.6 ± 77.8 | <0.0001 |
| Line tracing errors (number) | 28.9 ± 24.9 | 42.9 ± 34.2 | <0.0001 |
| Serial dotting test (seconds) | 64.7 ± 22.7 | 92.0 ± 44.2 | <0.0001 |
| PHES total score (+4 to −15, median IQR) | −2 (5.25) | −7 (9.00) | <0.0001 |
| ICT components (n = 175) | | | |
| Lures (number correct, max = 40) | 12.5 ± 8.9 | 14.4 ± 9.1 | 0.13 |
| Targets (% correct) | 96.1 ± 5.9 | 89.5 ± 14.5 | <0.0001 |
| Weighted lures (number) | 14.1 ± 11.4 | 22.4 ± 22.6 | <0.0001 |
| EncephalApp components (n = 125) | | | |
| Off Time (seconds) | 85.6 ± 24.6 | 103.0 ± 29.4 | 0.001 |
| On Time (seconds) | 102.7 ± 33.9 | 131.0 ± 51.3 | <0.0001 |
| Off Time + On Time (seconds) | 187.0 ± 52.6 | 234.0 ± 79.0 | <0.0001 |
| Off Time − On Time (seconds) | 12.5 ± 8.5 | 28.1 ± 27.4 | <0.0001 |
| Number of runs Off (number) | 5.7 ± 1.0 | 6.1 ± 1.5 | 0.02 |
| Number of runs On (number) | 6.3 ± 1.6 | 6.8 ± 4.0 | 0.28 |

Data is presented as mean ±SD unless mentioned otherwise.
Comparisons performed using unpaired t-tests or Mann-Whitney tests as appropriate.
CHE: covert hepatic encephalopathy,
ICT: inhibitory control test,
PHES: psychometric hepatic encephalopathy score.
A high score on Stroop components, ICT lures, ICT weighted lures and Digit symbol indicates poor performance and a low score in the remaining cognitive tests indicate poor performance.

TABLE 2
Details of patients with saliva collection

| | No prior Overt | Prior Overt HE | |
|---|---|---|---|
| | HE (n = 144) | (n = 123) | P value |
| Age (years) | 59.4 ± 6.6 | 60.6 ± 7.1 | 0.31 |
| Education (years) | 13.5 ± 2.5 | 13.6 ± 2.3 | 0.87 |
| Gender (male) | 51 (69%) | 42 (86%) | 0.05 |
| Proton Pump Inhibitor Use | 28 (38%) | 31 (63%) | 0.01 |
| Alcoholic etiology of cirrhosis | 21 (28%) | 15 (30%) | 0.52 |
| MELD score | 9.8 ± 6.5 | 15.6 ± 6.5 | <0.0001 |
| Lactulose | — | 47 | — |
| Rifaximin | — | 30 | — |
| CHE (yes/no) | | | |
| PHES (n = 122 total) | 13 (18%) | 29 (59%) | <0.0001 |
| ICT (n = 113 total) | 31 of 47 (66%) | 39 of 66 (59%) | 0.36 |
| Stroop (n = 70 total) | 24 of 42 (57%) | 23 of 28 (82%) | 0.03 |
| PHES components | | | |
| Number connection A (seconds) | 36.4 ± 20.1 | 52.6 ± 35.1 | 0.005 |
| Number connection B (seconds) | 91.6 ± 48.3 | 159.0 ± 116.0 | <0.0001 |
| Digit symbol (seconds) | 52.6 ± 20.0 | 42.3 ± 15.1 | 0.002 |
| Line tracing time (seconds) | 98.5 ± 38.2 | 112.9 ± 66.2 | 0.17 |
| Line tracing errors (number) | 29.7 ± 26.8 | 42.6 ± 30.2 | 0.02 |
| Serial dotting test (seconds) | 64.5 ± 24.4 | 81.1 ± 39.0 | 0.01 |
| PHES total score (+4 to −15, median IQR) | −1 (5.0) | −4 (7.5) | 0.005 |
| ICT components (n = 113) | | | |
| Lures (number correct, max = 40) | 12.1 ± 9.4 | 12.6 ± 8.2 | 0.78 |
| Targets (% correct) | 96.5 ± 5.8 | 91.6 ± 14.1 | 0.03 |
| Weighted lures (number) | 12.7 ± 10.4 | 20.1 ± 22.6 | 0.04 |
| EncephalApp components (n = 70) | | | |
| Off Time (seconds) | 88.2 ± 26.2 | 94.6 ± 32.3 | 0.04 |
| On Time (seconds) | 104.2 ± 32.1 | 117.5 ± 52.1 | 0.24 |
| Off Time + On Time (seconds) | 192.3 ± 56.4 | 212.0 ± 83.6 | 0.29 |
| Off Time − On Time (seconds) | 14.0 ± 9.1 | 22.9 ± 22.9 | 0.07 |
| Number of runs Off (number) | 5.9 ± 0.8 | 6.1 ± 1.6 | 0.12 |
| Number of runs On (number) | 6.3 ± 1.4 | 7.0 ± 2.8 | 0.23 |

Data is presented as mean ±SD unless mentioned otherwise.
Comparisons performed using unpaired t-tests or Mann-Whitney tests as appropriate.
CHE: covert hepatic encephalopathy,
ICT: inhibitory control test,
PHES: psychometric hepatic encephalopathy score.
A high score on Stroop components, ICT lures, ICT weighted lures and Digit symbol indicates poor performance and a low score in the remaining cognitive tests indicate poor performance.

### Covert HE Diagnosis:

In the entire group, using PHES, 145 (54%) of patients had CHE, in those given Stroop (total n=125), 102 (81%) had CHE, while in patients administered the ICT (n=175),115 (65%) had CHE (Table 3). In the 125 patients of these that underwent both

Stroop and PHES, 43 (34%) were discordant (p<0.0001 Chi-square). In the 175 patients that had both ICT and PHES, 70 (39%) were discordant (p=0.001) and in the 99 who had both Stroop and ICT, discordant results were seen in 29 (29%, p=0.01).

TABLE 3
Patients with CHE on PHES compared to those without CHE in all patients

| | Patients who provided stool | | | Subset who also provided saliva | | |
|---|---|---|---|---|---|---|
| | No covert | Covert | | No covert | Covert | |
| | HE | HE | | HE | HE | |
| | (n = 122) | (n = 145) | P value | (n = 78) | (n = 42) | P value |
| Age | 59.4 ± 9.1 | 60.1 ± 7.0 | 0.07 | 59.5 ± 6.1 | 61.4 ± 8.2 | 0.21 |
| (years) | | | | | | |
| Education | 13.8 ± 2.3 | 13.4 ± 2.2 | 0.20 | 13.8 ± 2.5 | 13.1 ± 2.1 | 0.10 |
| (years) | | | | | | |
| Gender | 85 | 122 | 0.003 | 54 | 38 | 0.006 |
| (male) | | | | | | |
| Proton | 54 | 77 | 0.11 | 36 | 22 | 0.07 |
| Pump | | | | | | |
| Inhibitor | | | | | | |
| use | | | | | | |
| MELD | 14.3 ± 7.1 | 11.3 ± 4.7 | <0.0001 | 15.7 ± 7.1 | 10.2 ± 3.9 | <0.0001 |
| score | | | | | | |
| Prior OHE | 42 | 82 | <0.0001 | 20 | 29 | <0.0001 |
| Lactulose | 38 | 80 | <0.0001 | 19 | 28 | 0.001 |
| Rifaximin | 22 | 55 | <0.0001 | 12 | 18 | <0.0001 |

Data is presented as mean ± SD unless mentioned otherwise. Comparisons performed using unpaired t-tests or Mann-Whitney tests as appropriate. OHE: overt hepatic encephalopathy

In patients without prior OHE who gave stool, there were 63 (of 144, 44%) that were positive for CHE on PHES, 54 (of 87, 62%) positive for ICT and 35 (of 49, 71%) positive for Stroop CHE. these patients only 61 patients were CHE positive and for both PHES and Stroop (Table 4). When CHE positivity was compared, 83 (58%) were discordant between ICT and PHES, 69 (47%) were discordant between Stroop and PHES and 54 (38%) were discordant between ICT and Stroop.

TABLE 4
Patients with CHE on PHES compared to those without CHE in those
without prior OHE

| | Patients who provided stool | | | Subset who also provided saliva | | |
|---|---|---|---|---|---|---|
| | No covert | Covert | | No covert | Covert | |
| | HE | HE | P | HE | HE | P |
| | (n = 80) | (n = 63) | value | (n = 58) | (n = 13) | value |
| Age | 59.2 ± 7.0 | 59.9 ± 7.5 | 0.58 | 65.0 ± 8.5 | 59.8 ± 6.44 | 0.06 |
| (years) | | | | | | |
| Education | 13.9 ± 2.4 | 13.6 ± 2.2 | 0.56 | 13.8 ± 2.5 | 13.1 ± 2.1 | 0.09 |
| (years) | | | | | | |
| Gender | 51 | 44 | 0.34 | 39 | 11 | 0.19 |
| (male) | | | | | | |
| Proton | 35 | 19 | 0.11 | 23 | 5 | 0.52 |
| Pump | | | | | | |
| Inhibitor | | | | | | |
| use | | | | | | |
| MELD | 10.3 ± 4.7 | 10.2 ± 4.5 | 0.93 | 9.3 ± 3.7 | 11.4 ± 3.8 | 0.09 |
| score | | | | | | |

Data is presented as mean ± SD unless mentioned otherwise. Comparisons performed using unpaired t-tests or Mann-Whitney tests as appropriate. OHE: overt hepatic encephalopathy

Ultimately, there was poor kappa agreement between the modalities (PHES vs ICT=0.15, PHES vs Stroop=0.35, Stroop vs ICT=0.20 for the diagnosis in those who had more than one testing modality used.

### Microbiota Changes

### Microbiota Analysis:

Stool samples were obtained from all 267 patients while saliva was obtained from 122 patients (49 prior OHE and 73 without OHE). The entire group was first evaluated from the salivary and stool microbiota perspective based on CHE on the three individual modalities.

We then evaluated MHE on PHES in patients without prior OHE on stool and same for ICT and Stroop. Shannon diversity indices are shown in Table 5.

TABLE 5
Diversity indices between groups

| | All patients | | Patients without prior OHE | |
|---|---|---|---|---|
| | CHE | No-CHE | CHE | No-CHE |
| Stool microbiota | | | | |
| diversity | | | | |
| CHE on PHES | 1.9 ± 0.7 | 2.2 ± 0.6* | 1.9 ± 0.7 | 2.2 ± 0.6* |
| CHE on ICT | 2.0 ± 0.7 | 2.1 ± 0.6 | 2.2 ± 0.6 | 2.1 ± 0.8 |
| CHE on Stroop | 1.9 ± 0.6 | 2.1 ± 0.6* | 1.7 ± 0.6 | 2.2 ± 0.6* |
| Salivary | | | | |
| microbiota | | | | |
| diversity | | | | |
| CHE on PHES | 2.0 ± 0.4 | 2.3 ± 0.4* | 2.0 ± 0.5 | 2.5 ± 0.3* |
| CHE on ICT | 2.1 ± 0.5 | 2.1 ± 0.5 | 2.2 ± 0.3 | 2.3 ± 0.3 |
| CHE on Stroop | 2.0 ± 0.5 | 2.3 ± 0.4* | 2.1 ± 0.4 | 2.2 ± 0.2 |

CHE: covert hepatic encephalopathy,
OHE: overt hepatic encephalopathy.
ICT: inhibitory control test,
PHES: psychometric hepatic encephalopathy score.
CHE diagnosis made according to US-based norms.

### LEFSe in the Entire Group:

### Stool Changes (FIG. 2A):

Using PHES as the definition of CHE, there was a higher relative abundance of Lactobacillales and Micrococcaceae with lower Lachnospiraceae, Clostridiales Incertae Sedis XI and XIII and Pasteurellaceae in those with CHE. Similarly, using ICT, there was a higher relative abundance of Enterobacteriaceae, Streptocococcaceae, Micrococcaceae and Eubacteriaceae and lower Bacteroidaceae in those with CHE. When Stroop was used here was a higher relative abundance of Lactobacillales with lower Eubacteriaceae, Telmatobacter and taxa belonging to Proteobacteria in those with CHE was noted.

### Salivary Changes (FIG. 2C):

In patients with CHE on PHES, salivary Lactobacillaceae, Streptococcaceae, Sutterellaceae and Clostridiaceae were higher while a lower relative abundance of Prevotellaceae, Saccharibacteria, Fusobacteriaceae and Eubacteriaceae was seen. Using ICT, again there was a higher relative abundance of Lactobacillaceae with lower Saccharibacteria, and constituents of Proteobacteria in those with CHE. On Stroop, in those with CHE again a higher relative abundance of Lactobacillaceae, Streptococcaceae and lower Proteobacterial, Fusobacteria and Prevotellaceae relative abundance was observed in those with CHE.

LEFSe in Patients without Prior OHE:

Stool changes (FIG. 2B): Patients with CHE on PHES had a higher Lactobacillaceae and Micrococcaceae and a lower relative abundance of Lachnospiraceae, Acidaminococcacaeae and Cyanobacteria compared to those without CHE. Using ICT, there were again higher Lactobacillaceae, Enterobacteriaceae and Streptococcaceae and lower Bacteroidaceae and Peptococcaceae in those with CHE. When Stroop was used, there was a higher relative abundance of Lactobacillaceae, Bifidobacteriaceae, Micrococcaeae and Gammaproteobacteria and lower Cyanobacteria and Clostridiales Cluster XIII in those with CHE.

### Salivary Changes (FIG. 2D):

PHES-associated CHE was associated with higher Lactobacillaceae and lower Proteobacteria, Chloroplast and several members of the Firmicutes phylum. Using Stroop, there was a higher relative abundance of Lactobacillaeae and Streptococcaceae and lower Proteobacteria in those with CHE. Patients with CHE on ICT had higher Veillonellacaeae and lower Proteobacteria, Staphylococcaceae and Acetobacteriaceae compared to those without CHE.

### Logistic Regression:

As shown in Table 6, several microbial families in the stool and saliva were associated with CHE independent of the clinical variables input. Specifically, Lachnospiraceae were associated with protection from CHE on PHES and Stroop while Veillonellaceae were associated with ICT- associated CHE. In the saliva, Streptococcaceae and Corobacteriaceae were associated with CHE on the three modalities while Clostridalies cluster XI and Prevotellaceae were associated with protection against CHE.

TABLE 6
Logistic regression results for stool and salivary microbiota at the family level

| | All patients | | Patients without prior OHE | |
|---|---|---|---|---|
| | Higher in CHE | Higher in no-CHE | Higher in CHE | Higher in no-CHE |
| Stool microbiota | | | | |
| and clinical variables | | | | |
| CHE on PHES | MELD, age, | Lachnospiraceae | MELD, age, | Lachnospiraceae |
| | male gender | | male gender | |
| CHE on ICT | Veillonellaceae | — | Veillonellaceae | — |
| CHE on Stroop | Male gender | Lachnospiraceae | Male gender | Lachnospiraceae |
| Salivary | | | | |
| microbiota | | | | |
| CHE on PHES | Age, prior OHE, | — | Lactobacillaceae | — |
| | MELD, | | | |
| | Streptococcaceae | | | |
| CHE on ICT | Coriobacteriaceae | — | Coriobacteriaceae | Clostridiales |
| | | | | clusterXI, |
| | | | | Aerococcaceae |
| CHE on Stroop | Streptococcaceae | — | Male Gender | Prevotellaceae |

CHE: covert hepatic encephalopathy,
OHE: overt hepatic encephalopathy.
ICT: inhibitory control test,
PHES: psychometric hepatic encephalopathy score.
CHE diagnosis made according to US-based norms.
Clinical variables studied were age, gender, MELD score, prior OHE (in the entire group), PPI use and education in years.

### Genus-Level Changes in Taxa Found on Logistic Regression:

The specific stool genera in Lachnospiraceae associated with protection from CHE as assessed by PHES were Blautia, Dorea, Roseburia, Clostridium XIVb, Robinsiella, Coprococcus and Ruminococcus. The stool genera associated with protection from CHE assessed using ICT were the genera Ruminococcus, Clostridium XIVb and Cellulosilyticum. The stool genera associated with protection from CHE when assessed using Stroop were Clostridium XIVb and Lachnospira. In the stool in patients only without prior OHE, the similar Lachnospiraceae genera were higher in those without CHE based on PHES and Stroop assessment, while Veillonella was the genus higher in ICT-associated CHE.

In saliva, Streptococcus was the genus associated with CHE associated with Stroop and PHES assessment in the entire group. In those without prior OHE, Lactobacillus and Paralactobacillus were higher in CHE determined by PHES and Prevotella was higher in those with CHE based on Stroop performance. In those without CHE determined by ICT Abiotrophia from Aeroccocaceae and Clostridium XIVb of Lachnospiraceae were in greater relative abundance.

### Discussion:

The current study results demonstrate that patients with cirrhosis and CHE defined according to specific cognitive assessment strategies have unique microbial signatures in the stool and saliva. These microbial changes are associated with the diagnosis of CHE independent of clinical criteria and the presence of specific bacterial taxa is indicative of normal cognition in this population.

Patients with cirrhosis and CHE suffer from a poor health-related quality of life, a greater likelihood to progress to OHE, increased need for hospitalization, and have a worse survival compared to those without CHE (22, 23). These patients are often difficult to diagnose due to logistic concerns related to the available cognitive assessment methodologies including the relatively poor diagnostic agreement between these tests (24). Consistent with the literature we found a similar discordance between PHES, Stroop and ICT in our patient population (4, 6).

These tests interrogate different parts of the brain with psychomotor speed being a common denominator between the tests. PHES places a strong demand on a subject's visual-motor coordination and abstraction ability (14). In contrast with the PHES the EncephalApp Stroop emphasizes cognitive flexibility; with the ICT assessing working memory and response inhibition (25, 26). Given that each of the three cognitive methods used in this paper emphases differing cognitive skill sets, it is not surprising that CHE classification, based on individual test results compared with locally derived norms, would vary (27).

An altered gut-liver-brain axis is believed to be a central pathogenic factor accounting for the spectrum of cognitive impairment in cirrhosis, and indeed most HE-related therapies are focused on the gut (2, 28). Therefore, the changes in brain function underlying impaired performances on these tests could be related to alterations in gut microbiota. Changes in microbiota in cirrhosis have been shown in the stool, intestinal mucosa, serum and saliva (7, 29-32). This is likely related to changes in underlying immune function in cirrhosis that allows these alterations in microbiota to occur (13, 33). Patients with cirrhosis, especially those with advanced cirrhosis and OHE, have lower relative abundance of autochthonous bacterial taxa and higher potentially pathogenic ones (11). These are also associated with salivary bacterial changes (19). Therefore, specific microbial signatures that were found in our study that are linked to specific microbiota are intriguing.

We found that Shannon diversity indices varied according to the test used and patients who were positive on PHES and Stroop, but not ICT, had lower diversity in the stool and salivary microbiota. This trend continued to a large extent in those without prior OHE. This could be a function of liver disease severity as patients with CHE on PHES in the entire group had a higher MELD score compared to the rest. However, there are likely other factors at play because the MELD score was similar in patients without prior OHE who were positive on PHES, and yet their diversity was lower. Regardless, the basic concept of diversity of the microbiota already showed differentiating features between the three modes of CHE diagnosis.

Specific bacterial taxa in the stool and saliva differentiated between patients with and without CHE, but Lactobacillaceae were over-represented in the CHE group (30). In the stool Lactobacillaceae are associated with the use of lactulose, which was prescribed to the majority of prior OHE patients. However, we found a higher relative abundance of Lactobacillaceae in the stool of patients without prior OHE who had CHE and in the saliva as well. Since patients without OHE were not treated with lactulose and were not on specific probiotics, it is highly unlikely that this was an iatrogenic change. In prior studies, Lactobacillaceae have been associated with more advanced liver disease and to be linked with ammonia-associated changes on brain MRI (9). In animal models of cirrhosis, lactic acid has been associated with cerebral edema (34). In the logistic regression both Lactobacillus and Paralactobacillus genera were found to be higher in saliva of patients with CHE due to PHES. The intriguing aspect is that this increase in relative abundance of Lactobacillaceae was greater in the group without prior OHE and was found to be higher in patients with CHE regardless of the modality used. While there have been studies evaluating the role of Lactobacillus-only probiotics in cirrhosis, they have not consistently resulted in cognitive improvement (35). Also, there are several species within the Lactobacillus genus that are associated with pathogenic outcomes in addition to being probiotic in nature (36). Lactobacillus spp have been also associated with super-infections with other organisms and can increase after immunosuppressive therapy (37, 38). While Lactobacillaceae changes did not remain significant after logistic regression, these relatively consistent differences are intriguing.

On the other hand, genera belonging to Lachnospiraceae were present in greater relative abundance in patients without CHE in the stool in those with PHES and Stroop, even on logistic regression independent of clinical factors. Specific genera included Blautia, Roseburia, Clostridium XIVb and Ruminococcus. These taxa are associated with intestinal barrier integrity and short-chain fatty acid production and are usually found in higher relative abundances in patients with good cognition in prior studies (31, 39, 40). Specific Clostridia are also responsible for synthesizing the neuro-protective 3-indole propionic acid as well (41). The interesting aspect is that the presence of these taxa especially Clostridium XIVb and Ruminococcus indicates better cognitive function on all three testing strategies and could be a method to exclude significant cognitive dysfunction in patients with cirrhosis.

In the saliva, apart from the Lactobacillaceae changes, there was a higher relative abundance of Veillonella and Streptococcus in patients with cognitive impairment, even on logistic regression. Streptoccocaceae are associated with production of ammonia via urease activity, which could be associated with cognitive dysfunction and were found in greater relative abundance when the entire group was studied rather than those without OHE (42). Interestingly, these oral-origin taxa were not consistently higher in the stool of patients with CHE, likely due to the similar proportion of PPI use across the groups with and without CHE (43).

The association of altered biological processes in the determination of brain dysfunction in cirrhosis is relevant from a clinical and patho-physiological perspective. In prior studies, PHES and EEG have been separately linked with systemic inflammation and ammonia metabolism in patients with cirrhosis (8). Both modalities were independently associated with poor outcomes regardless of the pathogenesis. There is published evidence that changes in microbial composition can be associated with hospitalizations, HE episodes, death and recovery of brain function post-transplant (44-47). Prior reports have also focused differentially on specific gut microbiota and associated ammonia-related and inflammation-related consequences on brain MRI in cirrhosis (9). A prior study from China has found a higher stool Streptoccoccae relative abundance linked with ammonia in patients with cognitive impairment on paper pencil tests (42). The current results extend these results further by including the entire spectrum of cognitive dysfunction, using multiple methods of cognitive impairment to define CHE and by using salivary microbiota.

We conclude that there is a specific microbial signature in the stool and salivary microbiota that is associated with individual cognitive impairment in patients with cirrhosis. These microbial changes are associated with cognitive impairment independent of clinical factors. Specific microbial taxa are associated with good cognitive function regardless of CHE testing modality and could potentially be used to circumvent CHE testing as a beneficial biomarker of a healthy gut-liver-brain axis in cirrhosis and other chronic diseases.

### REFERENCES

1. Stepanova et al. Clin Gastroenterol Hepatol 2016.
2. Vilstrup et al. Hepatology 2014; 60:715-35.
3. Bajaj et al. Aliment Pharmacol Ther 2011; 33:739-47.
4. Montagnese et al. World J Gastroenterol 2014; 20:15756-62.
5. Goldbecker et al. Gut 2013; 62:1497-504.
6. Lauridsen et al. Metab Brain Dis 2011; 26:135-9.
7. Iebba et al. Sci Rep 2018; 8:8210.
8. Montagnese et al. Hepatology 2011; 53:558-66.
9. Ahluwalia et al. Sci Rep 2016; 6:26800.
10. Shawcross et al. J Hepatol 2004; 40:247-54.
11. Bajaj et al. Nature 2015; 525:E1-2.
12. Bajaj et al. J Hepatol 2014; 60:940-7.
13. Albillos et al. J Hepatol 2014; 61:1385-96.
14. Weissenborn et al. J Hepatol 2001; 34:768-73.
15. Bajaj et al. Gastroenterology 2008; 135:1591-1600 el.
16. Bajaj et al. Clin Gastroenterol Hepatol 2014.
17. Allampati et al. Am J Gastroenterol 2016; 111:78-86.
18. Amodio et al. Gastroenterology 2010; 139:510-8, 518 e1-2.
19. Bajaj et al. Hepatology 2015; 62:1260-71.
20. Hamady et al. Genome Res 2009; 19:1141-52.
21. Segata et al. Genome Biol 2011; 12:R60.
22. Ampuero et al. Gastroenterology;149:1483-1489.
23. Patidar et al. Am J Gastroenterol 2014; 109:1757- 63.
24. Bajaj et al. Gastroenterology 2015; 149:1330-3.
25. Garavan et al. Proc Natl Acad Sci U S A 1999; 96:8301-6.
26. Pardo et al. Proc Natl Acad Sci U S A 1990; 87:256-9.
27. Felipo et al. World J Gastroenterol 2014; 20:11815-25.
28. Kang et al. Hepatology 2016; 64:1232-48.
29. Santiago et al. Sci Rep 2016; 6:25001.
30. Bajaj et al. Am J Physiol Gastrointest Liver Physiol 2012; 302:G168-75.
31. Bajaj et al. Am J Physiol Gastrointest Liver Physiol 2012; 303:G675-85.
32. Chen et al. Hepatology 2011; 54:562-72.
33. Patel et al. Hepatology 2015; 62:1001-3.
34. Bosoi et al. J Hepatol 2014; 60:554-60.
35. Bajaj et al. Aliment Pharmacol Ther2014; 39:1113-25.
36. Salminen et al. Clin Infect Dis 2004; 38:62-9.
37. Reynolds et al. Gut Microbes 2014; 5:522-32.
38. Xu et al. Microbiol Res 2015; 171:97-106.
39. Rios-Covian et al. Front Microbiol 2015; 6:825.
40. Morrison et al. Gut Microbes 2016; 7:189-200.
41. Wikoff et al. Proc Natl Acad Sci USA 2009; 106:3698-703.
42. Zhang et al. Am J Gastroenterol 2013; 108:1601-11.
43. Bajaj et al. Am J Gastroenterol 2018.
44. Bajaj et al. Sci Rep 2015; 5:18559.
45. Chen et al. J Gastroenterol Hepatol 2015.
46. Bajaj et al. Liver Transpl 2017; 23:907-914.
47. Bajaj et al. Clin Gastroenterol Hepatol 2018.

### Example 2

Posttraumatic stress disorder (PTSD) is associated with altered gut microbiota that modulates cognitive performance in veterans with cirrhosis Posttraumatic stress disorder (PTSD) in veterans with cirrhosis was associated with poor cognitive performance This was associated with lower gut microbial diversity in PTSD with higher pathobionts belonging to Enterococcus and Escherichia/Shigella and lower beneficial taxa belonging to Lachnospiraceaeae and Ruminococcaceae, with functional alterations despite accounting for prior hepatic encephalopathy, psychoactive drug use, or model for end-stage liver disease score. Given the suboptimal response to current therapies for PTSD, targeting the gut microbiota could benefit the altered gut-brain axis in these patients.

Chronic liver disease and cirrhosis are epidemic in the veteran population (13). Patients with cirrhosis have an altered gut-liver-brain axis that manifests itself as hepatic encephalopathy (HE) (1, 8). HE consists of a spectrum ranging from subclinical covert HE (CHE) and the clinically apparent overt HE (OHE) (52). HE is associated with altered gut microbial composition and function, impaired intestinal barrier, and systemic inflammation, which results in the neuroinflammation underlying its clinical symptoms (50). These negatively affect daily function and prognosis and can result in persistent impairment (11, 45). Chronic liver diseases and cirrhosis in veterans are most often associated with lifestyle and addiction related diseases such as substance and alcohol use disorders and obesity, which can be exacerbated by posttraumatic stress disorder (PTSD) (13, 27, 28, 51). PTSD is epidemic in the combat veteran population and is often associated with chronic liver disease and greater mortality from chronic liver disease (15, 16, 20).

PTSD is associated with varying degrees of autonomic hyperreactivity, depression, and anxiety, which can result in cognitive impairment even in the absence of chronic liver disease and cirrhosis (25). There is evidence of an altered gut-brain axis in animal and human studies with PTSD without concomitant cirrhosis (26, 33). A prior study shows that veterans with PTSD and cirrhosis have poorer cognitive function compared with model for end-stage liver disease (MELD)-balanced veterans with cirrhosis without PTSD (14). However, the impact of gut microbial change related to PTSD in the setting of cirrhosis is unclear. This may be relevant as a new target for therapy since current therapy success rates in PTSD are suboptimal.

Our aim was to determine changes in gut microbiota and cognitive ability in veterans with cirrhosis and PTSD compared with those without PTSD.

### Materials and Methods

We prospectively enrolled outpatient veterans with cirrhosis diagnosed using biopsy, radiological evidence, or endoscopic evidence of varices in chronic liver disease. All subjects provided informed consent. We excluded patients with active illicit drug or alcohol abuse within the last 3 mo defined by current urine drug screens and interviews per standard of care, those unable to provide consent or samples, with a minimental status exam <25, with bipolar disorder, seizures, schizophrenia, or on antipsychotic and antiseizure medications. We also excluded patients who were currently on or had been on absorbable antibiotics over the last 3 mo, which included patients on spontaneous bacterial peritonitis prophylaxis. Patients on chronic antidepressants and opioids were included provided their regimen was stable over 3 mo before enrollment. The protocol was approved by the Institutional Review Board at the McGuire VA Medical Center, and all patients provided written informed consent.

Demographics, cirrhosis (etiology, MELD score), and HE (prior OHE) were recorded. PTSD was diagnosed using validated Diagnostic and Statistical Manual of Mental Disorders-5 (DSM-V) criteria (51a), and the current treatments for this condition were recorded. We recorded a detailed 3-day dietary recall for all subjects. Serum was also collected for lipopolysaccharide binding protein (LBP) levels (ng/ml) that were analyzed using published techniques at Assaygate (Ijamsville, MD) (10). All subjects underwent three cognitive testing strategies that have been used for covert HE testing: the inhibitory control test (ICT), psychometric hepatic encephalopathy score (PHES), and block design test (BDT) (52). ICT is a test of psychomotor speed, response inhibition, and working memory with the output being lures and weighted lures (9, 22). A low lure and weighted lure number indicate good performance PHES is a composite of five tests, and the total SDs beyond healthy controls are calculated (53). A high score indicates good performance BDT is a test of nonverbal problem solving and visual spatial ability (35), and a low score indicates poor performance. Covert HE based on ICT and PHES was defined using published US-based criteria (2).
Microbiota. Stool was collected on the same date as the cognitive tests using published techniques on the same day (5). 16S rRNA microbiota analysis was performed using Multitag sequencing on an Ion Torrent PGM as previously published (24). Microbiota diversity was calculated using the Shannon Diversity Index, with a higher score indicating greater diversity, and individual taxa were compared using linear discriminant effect size (LEfSe) (49). Predicted function of microbiota using Phylogenetic Investigation of Communities by Reconstruction of Unobserved States (PiCRUST) was performed for all samples and compared between PTSD vs. no-PTSD and subgroups with and without prior OHE using LEfSe (32). Correlation networks were created separately for the four subgroups based on the presence or absence of PTSD and prior OHE between microbiota at the genus level and cognitive tests (41). Only those interactions that were P<0.05 and r>0.6 or <−0.6 were included in the final analysis.
Statistical analysis. We compared demographics, cirrhosis details, cognitive performance, and microbiota outputs 1) between patients with or without PTSD; 2) in those without prior OHE, again PTSD vs. no-PTSD; and 3) in those with prior OHE, PTSD vs. no-PTSD.

### Results

We enrolled 93 male veterans with cirrhosis, of whom 29 were diagnosed with PTSD. Of the total patient number, 32 had prior OHE, which was controlled on lactulose and rifaximin.
Clinical comparisons. As shown in Table 7, cirrhotic subjects with PTSD had similar demographics compared with those without PTSD. Cirrhosis severity, prior OHE, and etiologies of cirrhosis were also similar between groups. PTSD patients had greater use of psychoactive drugs. Cognitive testing on PHES was similar between groups, but PTSD patients had significantly worse performance on block design and ICT. All veterans were from the Vietnam era, and all had experienced active combat. All patients followed a nonvegetarian diet and were born and brought up on East Coast of the United States. There were 42 patients without OHE or PTSD, 19 with PTSD without OHE, 22 with OHE without PTSD, and 10 with both (Table 8). Patients with either PTSD or OHE or both were older, but their educational attainment was similar compared with those without PTSD or OHE. As expected, cirrhosis severity was greater in those with prior OHE, as was cognitive impairment on PHES and its subtests. On the other hand, block design and weighted lures were worse in patients with PTSD with/without OHE compared with those who did not have PTSD or OHE. As reported before, covert HE prevalence was higher in those with prior OHE compared with those without on ICT (65 vs. 25%, P=0.001) and PHES (45 vs. 29%, P=0.001), but did not differ between those with and without PTSD on ICT (58 vs. 54%, P=0.76) or PHES (39 vs. 35%, P=0.71) (14). Psychoactive medication prevalence was higher in the PTSD subgroups regardless of OHE. No dietary differences between groups were seen (Tables 7 and 8). PTSD details. PTSD was diagnosed using DSM-V criteria, and in all cases was combat related. PTSD in affected patients was treated using psychotherapy in the minority and psychotropic medication in the rest. Of the 15 patients on psychoactive medications, the majority were on antidepressants (trazodone n=6, bupriopion n=4, sertraline n=3, citalopramn=2, fluoxetine n=2, and mirtazapine n=1) followed by benzodiazepines (n=2). Five patients were on more than one medication. The remaining patients who were not actively treated for PTSD specifically were not on disability at the time of testing and were able to complete the testing and analysis. In the 12 patients without PTSD that were on psychoactive medications, the major ones were anti-depressants (buproprion n=3, trazodone n=2, duloxetine n=1, fluoxetine n=1, venlafaxine n=1, sertraline n=1, citalopram n=1) followed by benzodiazepines (n=2).

TABLE 7
Comparison between PTSD and no-PTSD groups as a whole

| | No-PTSD (n = 64) | PTSD (n = 29) |
|---|---|---|
| Age, yr | 58.8 +/− 8.2 | 59.9 +/− 8.4 |
| Education, yr | 13.4 +/− 2.4 | 13.1 +/− 2.7 |
| Total calories/day | 2,513 +/− 396 | 2,489 +/− 451 |
| Percent calories from | 16 +/− 8 | 15 +/− 9 |
| protein/day | | |
| Lipopolysaccharide-binding | 9.4 +/− 2.9 | 12.7 +/− 4.3* |
| protein, ng/ml | | |
| MELD score | 12.0 +/− 7.5 | 10.5 +/− 7.9 |
| Etiology (HCV, alcoholic, | 28/12/7/10/7 | 10/10/4/3/2 |
| Alc+ HCV, NASH, others) | | |
| Prior overt HE | 22 (34%) | 10 (34%) |
| Past alcohol use disorder | 40 (63%) | 19 (66%) |
| PTSD Details | | |
| Current psychotherapy | 0 (0%) | 3 (10%) |
| Current psychoactive | 12 (19%) | 15 (51%)* |
| medications | | |
| Cognitive Testing | | |
| Number connection-A | 46.2 +/− 22.1 | 49.5 +/− 27.3 |
| Number connection-B | 130.0 +/− 75.6 | 153.0 +/− 106.0 |
| Digit symbol | 46.1 +/− 16.2 | 45.1 +/− 14.9 |
| Serial dotting | 80.4 +/− 36.1 | 75.7 +/− 33.1 |
| Line tracing time | 116.5 +/− 67.9 | 114.2 +/− 42.5 |
| Line tracing errors | 36.9 +/− 33.8 | 36.8 +/− 33.6 |
| PHES total | −3.7 +/− 3.4 | −4.2 +/− 4.1 |
| Block design test | 27.0 +/− 13.0 | 22.0 +/− 11.9* |
| Weighted lures | 15.0 +/− 11.0 | 22.1 +/− 13.9* |
| Shannon Diversity Index | 2.5 +/− 0.5 | 2.1 +/− 0.5* |

Values are means +/− SE;
n, number of patients.
PTSD, posttraumatic stress syndrome;
PHES, psychometric hepatic encephalopathy score;
HE, hepatic encephalopathy;
MELD, model for end-stage liver disease;
HCV, hepatitis C virus;
NASH, nonalcoholic steatohepatitis.
*P < 0.05 on unpaired t test or Man-Whitney as appropriate between groups;
high score on digit symbol and block design tests indicates good performance, while low score on the rest indicates good performance.
Higher Shannon diversity score indicates greater microbial diversity.

TABLE 8
Comparison between subgroups divided according to PTSD and prior OHE

| | | PTSD | OHE | | |
|---|---|---|---|---|---|
| | No-PTSD | Without | Without | PTSD | |
| | or OHE | OHE | PTSD | and OHE | P Value All |
| | (n = 42) | (n = 19) | (n = 22) | (n = 10) | Subgroups* |
| Age, yr | 55.8 +/− 8.3 | 60.6 +/− 6.2 | 60.6 +/− 7.3 | 60.4 +/− 5.7 | 0.03 |
| Education, yr | 13.7 +/− 1.9 | 12.4 +/− 2.9 | 14.3 +/− 2.6 | 13.2 +/− 2.1 | 0.10 |
| Total calories/day | 2,589 +/− 401 | 2,480 +/− 405 | 2,390 +/− 327 | 2,510 +/− 297 | 0.28 |
| Percent calories from | 17 +/− 6 | 16 +/− 9 | 16 +/− 7 | 15 +/− 5 | 0.57 |
| protein/day | | | | | |
| Lipopolysaccharide-binding | 8.1 +/− 2.3 | 9.9 +/− 3.1 | 11.7 +/− 3.1 | 14.3 +/− 5.1 | 0.005 |
| protein, ng/ml†‡ | | | | | |
| MELD score† 15.4 +/− 5.4 | 10.3 +/− 4.5 | 8.7 +/− 2.1 | 18.6 +/− 8.7 | 15.4 +/− 5.4 | <0.0001 |
| Etiology (HCV, alcoholic, | 21/5/3/7/6 | 7/5/4/2/1 | 7/7/4/3/1 | 3/5/0/1/1 | 0.09 |
| Alc + HCV, NASH, others) | | | | | |
| Past alcohol-use disorder | 23 (54%) | 13 (68%) | 17 (77%) | 6 (60%) | 0.27 |
| PTSD Details | | | | | |
| Current psychotherapy | | 2 (11%) | | 1 (10%) | 1.0 |
| On psychoactive | 9 (14%) | 10 (51%) | 3 (14%) | 5 (50%) | 0.006 |
| medications‡ | | | | | |
| Cognitive Testing | | | | | |
| Number connection-A† | 37.6 +/− 12.7 | 45.4 +/− 28.4 | 60.9 +/− 25.2 | 61.8 +/− 29.3 | <0.0001 |
| Number connection-B† | 106.9 +/− 47.2 | 146.7 +/− 114.3 | 171.2 +/− 98.5 | 173.0 +/− 93.1 | 0.01 |
| Digit symbol† | 52.8 +/− 14.5 | 47.8 +/− 12.8 | 33.8 +/− 11.0 | 38.8 +/− 17.6 | <0.0001 |
| Serial dotting† | 70.1 +/− 27.9 | 64.0 +/− 22.6 | 102.1 +/− 41.3 | 96.5 +/− 41.1 | <0.0001 |
| Line tracing time† | 98.8 +/− 41.1 | 99.5 +/− 38.4 | 152.3 +/− 91.8 | 142.0 +/− 91.8 | 0.002 |
| Line tracing errors | 31.1 +/− 31.2 | 40.8 +/− 39.9 | 47.6 +/− 35.6 | 28.3 +/− 13.7 | 0.24 |
| PHES total† | −3.1 +/− 3.3 | −3.8 +/− 4.1 | −8.6 +/− 4.3 | −7.4 +/− 5.4 | <0.0001 |
| Block design test | 30.0 +/− 13.7 | 22.6 +/− 12.7 | 20.0 +/− 8.9 | 22.6 +/− 10.2 | 0.01 |
| Weighted lures | 15.2 +/− 11.0 | 22.3 +/− 13.6 | 22.4 +/− 19.2 | 22.3 +/− 14.3 | 0.05 |
| Shannon Diversity Index† | 2.5 +/− 0.5 | 2.3 +/− 0.6 | 2.0 +/− 0.8 | 1.7 +/− 0.5 | 0.001 |

Values are means +/− SE;
n, number of patients.
PTSD, posttraumatic stress syndrome;
PHES, psychometric hepatic encephalopathy score;
OHE, prior overt hepatic encephlopathy;
MELD, model for end-stage liver disease;
HCV, hepatitis C virus;
NASH, nonalcoholic steatohepatitis.
*P < 0.05 ANOVA or Kruskal-Wallis tests.
†P < 0.05 between OHE vs. no-OHE groups.
‡P < 0.05 between PTSD vs. no-PTSD groups;
high score on digit symbol and block design tests indicates good performance, while low score on the rest indicates good performance.
Higher Shannon diversity score indicates greater microbial diversity

Microbiota changes. For Shannon diversity scores, in the entire group, advancing disease was associated with lower diversity. The MELD score was negatively correlated (r=0.54, P<0.0001), and there was a lower Shannon in those with prior OHE vs. the rest (1.94+/−0.70 vs. 2.45+/−0.55, P<0.001). Shannon was also correlated with PHES score (r=0.33, P<0.002), Weighted lures (r=−0.3, P<0.04), and block design (r=0.25, P<0.05).

PTSD was associated with a lower Shannon (Table 7), which was lowest in the group with prior OHE and PTSD (Table 8). Diversity scores were statistically similar between patients with an alcoholic etiology/not (2.4+/−0.6 vs. 2.4+/−0.4, P<0.58), hepatitis C virus (HCV) etiology/not (2.2+/−0.7 vs. 2.4+/−0.6. P<0.52), or those on psychoactive medications or not (2.4+/−0.6 vs. 2.3+/−0.7, P<0.17).

We performed a linear stepwise regression with Shannon diversity as the dependent variable using age, education, MELD score, prior OHE, PTSD, HCV etiology, and prior alcohol use as the potential predictors. The MELD score (coefficient −0.054, P<0.001) and PTSD (coefficient −0.28, P<0.02) were independently associated with Shannon diversity scores.

For individual microbiota differences, our primary comparisons were between PTSD vs. no-PTSD in those with prior OHE and those without prior OHE on LEfSe, which are shown in Table 9. Regardless of prior OHE, PTSD patients had lower relative abundance of potentially beneficial taxa belonging to families Ruminococcaceae and Lachnospiraceae. In addition, PTSD patients without prior OHE had higher relative abundance of potentially pathogenic taxa belonging to Enterococcaceae and Streptococcaceae.

TABLE 9
LEfSe changes in stool of patients with cirrhosis based on PTSD
and prior overt hepatic encephalopathy

| | Family_ Genus | |
|---|---|---|
| | Higher in PTSD | Higher in No-PTSD |
| Without prior OHE | Enterococcaceae_ | Lachnospiraceae_ |
| | Pillibacter | Ruminococcus |
| | Streptococcaceae_ | Lachnospiraceae_ |
| | Streptococcus | Roseburia |
| | Acidaminococcaceae_ | Lachnospiraceae_ |
| | Acidaminococcus | Anaerostipes |
| | Gracilibacteriaceae_ | Lachnospiraceae_ |
| | Lutispora | ClostridiumXIVa |
| | Clostridiaceae_ | Lachnospiraceae_ |
| | Proteiniclasticum | Eisenbergiella |
| | Thermodesulfobiaceae_ | Lachnospiraceae_ |
| | Thermodesulfobium | Lachnospira |
| | | Ruminococcaceae_ |
| | | Pseudoflavonibacter |
| With prior OHE | | Ruminococcaceae_ |
| | | Subdoligranulum |

Higher in one group means lower in the other and vice versa based on linear discriminant analysis score effect size (LEfSe).
OHE, overt hepatic encephalopathy;
PTSD, posttraumatic stress syndrome.

PiCRUST analysis. Tables 10, 11, and 12 show comparisons between groups with/without PTSD as a whole, without prior OHE, and with prior OHE, respectively. Correlation network analysis. We created subnetworks based on potentially beneficial taxa related to Ruminococcaceae Fecalibacterium, and other potential negative taxa, such as Enterococcaceae and Enterobacteriaceae constituents with other microbiota and cognitive tests in patients with PTSD with/without OHE, and similarly for those without PTSD (FIG.

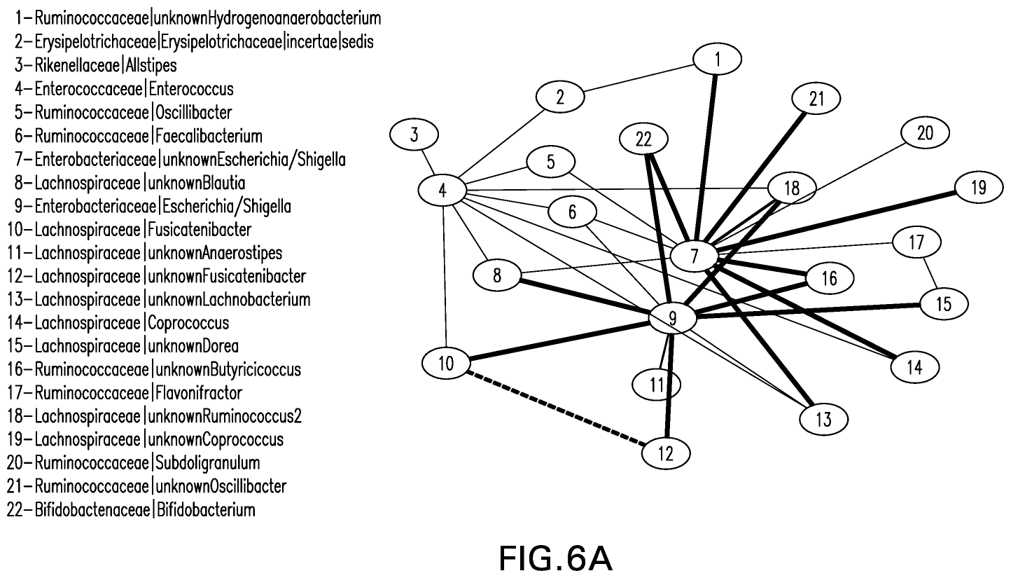

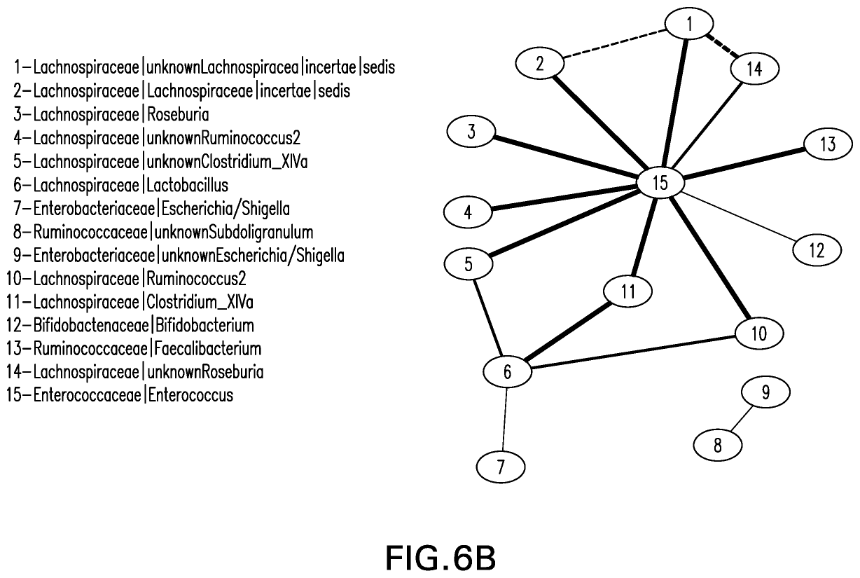

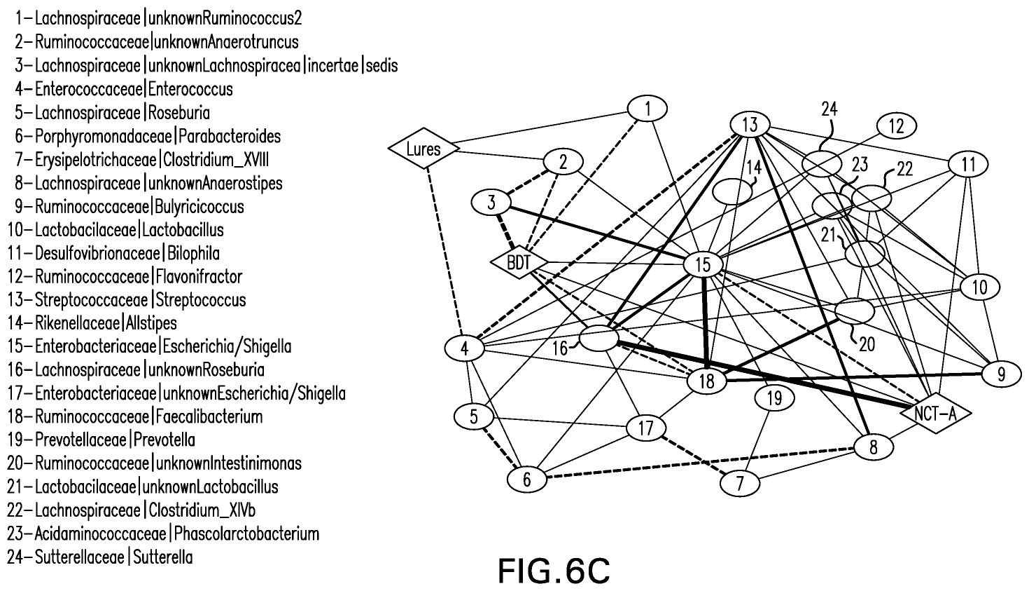

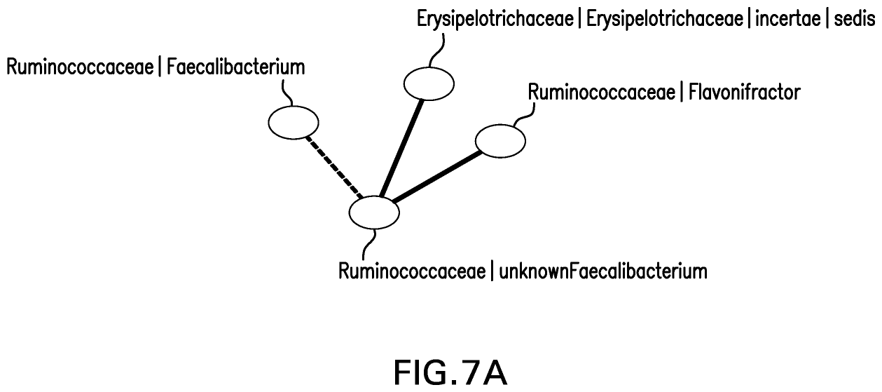

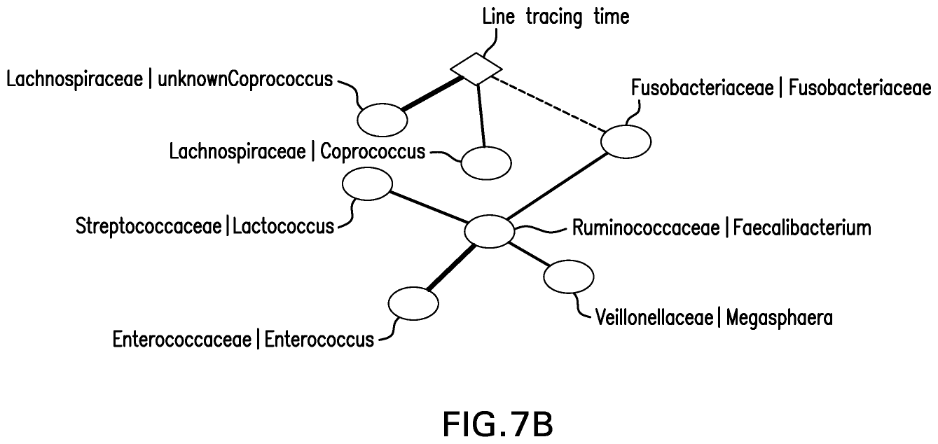

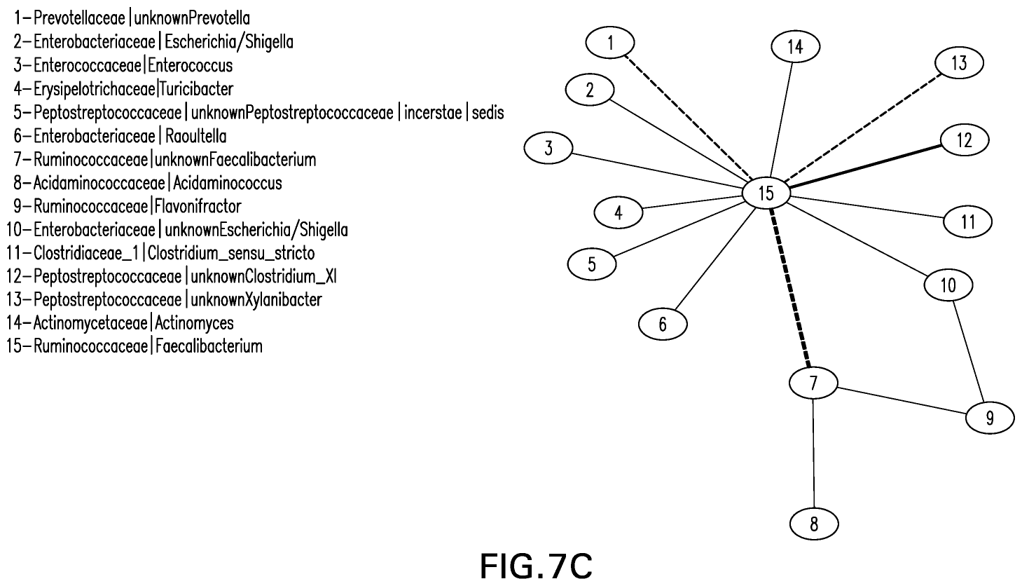

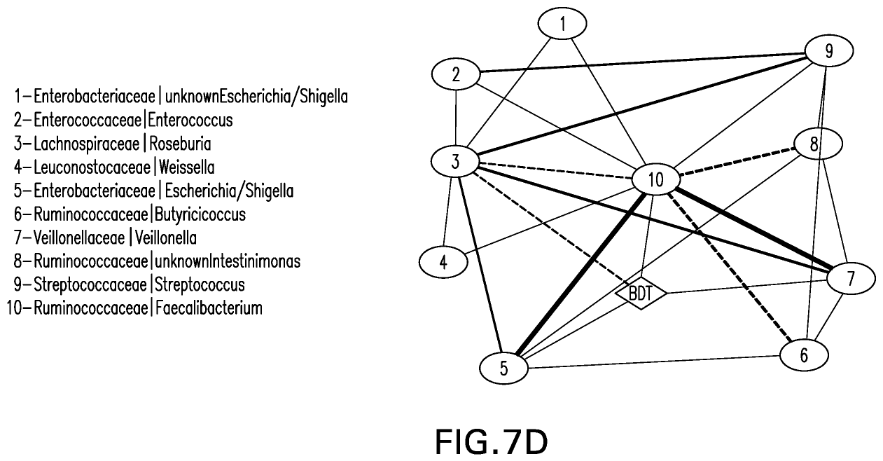

1, A-C). Regardless of subgroup, Fecalibacterium was positively correlated with other beneficial taxa and good performance on cognitive tests, especially in groups with prior OHE (FIG. 7, A-D). In patients without OHE or PTSD, there were no significant correlations between Shigella/Escherichia and Enterococcus and other bacteria and cognitive tests. Regardless of whether patients had OHE or not, PTSD patients demonstrated a significant positive correlation between Shigella/Escherichia and Enterococcus and a negative correlation between them and taxa belonging to Lachnospiraceae and Ruminococcaceae (FIG. 6, A and C). When patients without PTSD with OHE were studied, Enterobacteriaceae constituents were not prominent; rather, Enterococcus was associated with poor cognition and negatively with taxa belonging to Lachnospiraceae and Ruminococcaceae (FIG. 6B).

TABLE 10
PiCRUST values in all patients

| | Higher in Group | LDA |
|---|---|---|
| Phenyl-propanoid biosynthesis | No-PTSD | 2.35 |
| Cyano amino acid metabolism | No-PTSD | 2.31 |
| Terpenoid biosynthesis | PTSD | 2.21 |
| Alanine metabolism | PTSD | 2.07 |
| Genetic Information Processing: | PTSD | 2.03 |
| nucleotide excision repair | | |
| Genetic Information Processing: | PTSD | 2.18 |
| homologous recombination | | |
| Genetic Information Processing: | PTSD | 2.04 |
| base excision repair | | |
| Genetic Information Processing: | PTSD | 2.39 |
| ribosome biogenesis | | |
| Genetic Information Processing: | PTSD | 2.57 |
| DNA repair and recombination proteins | | |

PiCRUST, Phylogenetic Investigation of Communities by Reconstruction of Unobserved States.
Higher linear discriminant analysis (LDA) score indicates greater difference between the groups.

TABLE 11
PiCRUST values in patients without prior OHE

| | Higher in Group | LDA |
|---|---|---|
| Glyoxylate and dicarboxylate metabolism | No-PTSD | 2.11 |
| Inorganic ion transport and metabolism | No-PTSD | 2.04 |
| Phenyl-propanoid biosynthesis | No-PTSD | 2.21 |
| Arginine and proline metabolism | No-PTSD | 2.49 |
| Pentose and glucuronate interconversions | No-PTSD | 2.55 |
| Starch and sucrose metabolism | No-PTSD | 2.50 |
| Sphingolipid metabolism | No-PTSD | 2.49 |
| Oxidative phosphorylation | No-PTSD | 2.34 |
| Cyano amino acid metabolism | No-PTSD | 2.26 |
| Tyrosine metabolism | PTSD | 2.06 |
| Terpenoid biosynthesis | PTSD | 2.14 |
| Peptidoglycan biosynthesis | PTSD | 2.24 |
| Alzheimer's disease | PTSD | 2.01 |
| Genetic Information Processing: homologous | PTSD | 2.15 |
| recombination | | |
| Genetic Information Processing: DNA repair | PTSD | 2.65 |
| and recombination proteins | | |
| Genetic Information Processing: ribosome | PTSD | 2.47 |
| biogenesis | | |
| Membrane transport secretion system | PTSD | 2.65 |
| Alanine metabolism | PTSD | 2.03 |

PiCRUST, Phylogenetic Investigation of Communities by Reconstruction of Unobserved States.
Higher linear discriminant analysis (LDA) score indicates greater difference between the groups.

TABLE 12
PiCRUST values in patients with prior overt HE

| | Higher in Group | LDA Score |
|---|---|---|
| Flagellar assembly | No-PTSD | 2.94 |
| Bacterial chemotaxis | No-PTSD | 2.85 |
| Bacterial motility proteins | No-PTSD | 3.20 |
| Linoleic acid metabolism | PTSD | 2.44 |
| Glycan degradation | PTSD | 2.98 |
| Cellular antigen processing | PTSD | 2.76 |
| Alanine, aspartate, and glutamate metabolism | PTSD | 2.89 |
| Sphingolipid metabolism | PTSD | 2.80 |
| Glutamatergic synapse | PTSD | 2.50 |
| Alzheimer's disease | PTSD | 2.84 |

PiCRUST, Phylogenetic Investigation of Communities by Reconstruction of Unobserved States.
Higher linear discriminant analysis (LDA) score indicates greater difference between the groups.

### Discussion

The current study demonstrates more severe cognitive impairment in cirrhotic veterans with PTSD than those without PTSD. This is accompanied by a reduced microbial diversity, increased relative abundance of pathobionts belonging to Enterococcus and Proteobacteria, and a reduction in taxa belonging to Ruminococcaceae and Lachnospiraceae in PTSD patients with cirrhosis compared to cirrhosis patients without PTSD. Changes in the microbiota are linked with cognitive dysfunction, especially in those with prior OHE.

PTSD is a major contributor toward morbidity and mortality in veterans, especially related to chronic liver disease (20). Therefore, the current study results demonstrating greater dysbiosis and cognitive impairment in patients with PTSD regardless of prior OHE, can help us understand gut microbial modulation as a target in future studies. Replicating prior studies, there was additional cognitive impairment and psychoactive drug use in veterans with PTSD (14, 25). However, the groups were statistically similar with respect to cirrhosis severity, prior OHE, and demographic data. Given the major impact of prior OHE and its therapy on the gut-liver-brain axis, we split the comparison by OHE and PTSD and despite that found that greater impairments in the PTSD groups compared with their non-PTSD counterparts persisted.

The gut-brain axis has been implicated in the pathogenesis and progression of several diseases including depression, schizophrenia, autism, and HE (17). In a small human study focusing on non-combat PTSD, there was a lower relative abundance of Actinobacteria, Lentisphaerae, and Verrucomicrobia phyla in PTSD compared with trauma-exposed controls. The current study extends these into combat-related PTSD in male veterans and found major differences at the genus level that was independent of the stage of liver disease and prior OHE. We found a significant reduction in the diversity in PTSD patients, which also persisted when the patients without OHE were analyzed separately. PTSD was an independent predictor of lower diversity despite controlling for cirrhosis severity, OHE, alcohol, and psychoactive drugs. The alterations in microbiota found in our study were lower relative abundance of autochthonous genera belonging to Lachnospiraceae and Ruminococcaceae and higher relative abundance of pathobionts such as Enterococcus and Escherichia/Shigella in patients with PTSD. The autochthonous taxa belong to Firmicutes, which in rodent studies were reduced after intruder exposure in a population of male mice (23). Lachnospiraceae and Ruminococcaceae contain genera that produce short-chain fatty acids, strengthen the intestinal barrier, and are associated with better outcomes in patients with cirrhosis and other gut-based diseases (3, 42). These findings underline the importance of microbiota in PTSD, although the effect of concomitant liver disease remains unclear.

There is currently an interest in the neural and molecular mechanisms responsible for extinction of learned fear, particularly since fear extinction is a translationally relevant animal model for the treatment of human disorders, such as PTSD (38, 44). Previous studies suggest that fear extinction does not erase the original fear memory, but rather promotes the formation of a new inhibitory memory that reduces fear to the conditioned stimulus. Research in rodent models and humans suggests that the main structures are involved in processes related to fear extinction, particularly prefrontal and hippocampal inputs to the amygdala (38, 44). In support of this theoretical model, functional imaging studies of PTSD patients demonstrate hypoactivity in the ventromedial prefrontal cortex, suggesting that, at least in part, frontal region impairment may account for symptoms of PTSD (30). Consistent with this hypothesis, in our study patients with PTSD performed poorly on block design (assessing nonverbal problem-solving) and the ICT (evaluating the integrity of the frontal lobe inhibitory circuits and working memory). Performance on both of these measures depends on frontal/parietal integrity (35). These data suggest that PTSD, and the associated microbial change, selectively impacts frontal/parietal region function.

Chronic liver disease is a major consequence of lifestyle factors such as substance use and obesity that leads to hepatitis C, nonalcoholic fatty liver disease, and alcoholic liver disease (48). These lifestyle factors are associated with PTSD and depression, which often coexist and worsen the prognosis of these patients (25). In most patients with cirrhosis, cognitive impairment and gut microbial alterations are assumed to be related to covert or overt HE (52). However, as our current and prior findings show, PTSD adds to the cognitive impairment and alteration in microbial composition and diversity even in the skewed background of cirrhosis and prior OHE (14). When the correlation networks were analyzed, pathobionts Enterococcus and Esherichia/Shigella were negatively associated with good cognition and Lachnospiraceae/Ruminococcaceae constituents in patients with PTSD regardless of prior OHE. Moreover, patients without PTSD or OHE did not have a rich correlation network between microbiota and cognitive impairment, again demonstrating the additive impact of PTSD on microbial composition and interactions. These observations were strengthened by the higher levels of LBP, a functional correlate of excess gram-negative taxa such as Esherichia/Shigella, which was higher in PTSD patients, especially those with OHE. In addition to the differential correlation patterns in patients with and without PTSD, predicted functionality using PiCRUST showed that patients with PTSD as a whole had greater microbial functionality related to Alzheimer's disease, alanine, and terpenoids along with multiple levels of genetic information-processing functions. Terpenoids, which are usually secondary bile acids, whose excess presence is associated with intestinal barrier disruption, were higher in PTSD patients (19). On the other hand, indolic tryptophan derivatives, phenylpropanoids, which are associated with a functioning gut barrier and beneficial gut-brain effects, were higher in patients without PTSD (12, 46). A healthy gut barrier is also supported by arginine and proline metabolism, which were also higher in patients without PTSD (21). Mirroring the relatively higher Escherichia/Shigella and serum LBP, patients with PTSD also had higher predicted microbial functions related to glycan metabolism that is associated with endotoxin and gram-positive cell wall production. Additionally, there was also a higher excitatory amino acid glutaminergic synapse function association with PTSD-microbiota, which could relate to the anxiety present in these patients (29).

The gut-brain axis in cirrhosis has been modulated beneficially using probiotics, antibiotics such as rifaximin, laxatives such as lactulose, and a fecal microbiota transplant (FMT) (52). In those with prior OHE, however, despite lactulose and rifaximin being on board, our PTSD subjects still demonstrated increased Enterococcus and Proteobacteria and lower Ruminococcacaeae and Lachnospiracaeae compared with OHE without PTSD. This shows that perhaps in the OHE setting, further therapeutic options may be needed to alleviate PTSD. FMTs from mice which had a “defeat” PTSD phenotype were able to transmit inflammatory processes and their depression-like behavior to naive mice, indicating that the microbiota play a major role in behavior (43). Prior FMT studies in cirrhosis have demonstrated improvement in brain dysfunction in those with prior OHE (7, 10). In addition, microbiota from post-FMT humans with cirrhosis were able to quench neuroinflammation engendered by the microbiota before FMT (37). The therapy for PTSD currently consists of group or individual psychotherapy and medications to focus on specific symptoms, such as depression, anxiety, or flashbacks. However, despite these efforts, the disability and prevalence of noncombat PTSD continues to be a major burden, and many veterans with PTSD are not able to function to maintain competitive employment (31). Superimposed chronic liver disease makes these disabilities worse (47). Our findings related to altered microbial composition even in the earlier cirrhosis stages that separated PTSD from non-PTSD veterans could be another target for therapy (18). Specifically, we found that genera such as Fecalibacterium were positively related to good cognition, other autochthonous taxa, and negatively with pathobionts regardless of PTSD or prior OHE. Fecalibacterium and other members of Ruminococaceae have been associated with a strong intestinal barrier, are lower in those with depression, and could therefore be linked with better outcomes if studied as “psychobiotics” (18, 36, 39).

We conclude that in male veterans with cirrhosis, combat-related PTSD is associated with cognitive impairment, lower microbial diversity, greater relative abundance of pathobionts such as Escherichia/Shigella, and lower relative abundance of beneficial gut microbial taxa such as Fecalibacterium. This was associated with functional changes in the gut microbiota with implications for the gut-brain axis. Microbial changes were independent of prior overt hepatic encephalopathy and represent a new target to benefit veterans with cirrhosis and PTSD.

### REFERENCES

1. Ahluwalia et al. Sci Rep 6: 26800, 2016. doi:10.1038/srep26800.
2. Allampati et al. Am J Gastroenterol 111: 78-86, 2016. doi:10.1038/ajg.2015.377.
3. Bajaj et al. Nature 525: E1-E2, 2015. doi:10.1038/ nature14851.
4. Bajaj et al. Hepatology 62: 1260-1271, 2015.doi:10.1002/hep.27819.
5. Bajaj et al. PLoS One 8: e60042, 2013. doi:10.1371/journal.pone. 0060042.
6. Bajaj et al. Hepatology 66: 1727-1738, 2017. doi:10.1002/hep.29306.
7. Bajaj et al. Am J Physiol Gastrointest Liver Physiol 302: G168-G175, 2012. doi:10.1152/ajpgi.00190.2011.
8. Bajaj et al. Am J Gastroenterol 102: 754-760, 2007. doi:10.1111/j.1572-0241.2007.01048.x.
9. Bajaj et al. Hepatology hep.30690, 2019. doi:10.1002/hep.30690.
10. Bajaj et al. Gastroenterology 138: 2332-2340, 2010. doi:10.1053/j.gastro.2010.02.015.
11. Bansal et al. Proc Natl Acad Sci USA 107: 228-233, 2010. doi:10.1073/pnas.0906112107.
12. Beste et al. Gastroenterology 149: 1471-1482.e5, 2015. doi:10.1053/j.gastro.2015.07.056.
13. Burroughs et al. Dig Dis Sci 63: 481-485, 2018. doi:10.1007/s10620-017-4894-2.
14. Cameron et al. Mil Med Res 6: 8, 2019. doi:10.1186/s40779-019-0198-5.
15. Charlson et al. Lancet 394: 240-248,2019. doi:10.1016/50140-6736(19)30934-1.
16. Cryan et al. Can J Psychiatry 61: 201-203, 2016. doi:10.1177/0706743716635538.
17. Dinan et al. Biol Psychiatry 74: 720-726, 2013. doi:10.1016/j.biopsych.2013.05.001.
18. Donia et al. Science 349: 1254766, 2015. doi:10.1126/science.1254766.
19. Forehand et al. Am J Prey Med 57: 145-152, 2019. doi:10.1016/j.amepre.2019.03.014.
20. Fritz Infect Immun 81: 3500-3502, 2013. doi:10.1128/IAI.00789-13.
21. Garavan et al. Proc Natl Acad Sci USA 96: 8301-8306, 1999. doi:10.1073/pnas.96.14.8301.
22. Gautam et al. J Neurosci Res 96: 1311-1323, 2018. doi:10.1002/jnr.24229.
23. Hamady et al. Genome Res 19: 1141-1152, 2009. doi:10.1101/gr.085464.108.
24. Hayes et al. Front Integr Nuerosci 6: 89, 2012. doi:10.3389/fnint.2012.00089.
25. Hemmings et al. Psychosom Med 79: 936 -946, 2017. doi:10.1097/PSY.0000000000000512.
26. Kanwal et al. Gastroenterology 140:1182-1188.el, 2011. doi:10.1053/j.gastro.2010.12.032.
27. Kanwal et al. Clin Gastroenterol Hepatol 14: 301-308.e2, 2016. doi:10. 1016/j.cgh.2015.08.010.
28. Kelmendi et al. Eur J Psychotraumatol 7: 31858, 2016. doi:10.3402/ejpt.v7.31858.
29. Koenigs et al. Neuroscientist 15: 540-548, 2009. doi: 10.1177/1073858409333072.
30. Kunst J Interpers Violence 26: 377-393, 2011. doi:10.1177/0886260510362894.
31. Langille et al. Nat Biotechnol 31:814-821, 2013. doi:10.1038/nbt.2676.
32. Leclercq et al. Can J Psychiatry 61: 204-213, 2016. doi:10.1177/0706743716635535.
33. Leclercq et al. Proc Natl Acad Sci USA 111: E4485-E4493, 2014. doi:10.1073/pnas.1415174111.
34. Lezak, Neuropsychological Assessment. New York: Oxford University Press, 2012.
35. Liang et al. Int J Mol Sci 19: E1592, 2018. doi:10.3390/ijms19061592.
36. Liu et al. Hepatology hep.30827, 2019. doi:10.1002/hep.30827.
37. Milad et al. Nature 420: 70 -74, 2002. doi:10.1038/ nature01138.
38. Miguel et al. Curr Opin Microbiol 16: 255-261, 2013. doi:10.1016/j.mib.2013.06.003.
39. Mutlu et al. Am J Physiol Gastrointest Liver Physiol 302:G966-G978, 2012. doi:10.115²/ₐjpgi.00380.2011.
40. Naqvi et al. Chem Biodivers 7: 1040-1050,2010. doi:10.1002/cbdv.200900324.
41. Nava et al. Gut Microbes 2: 99-104, 2011. doi:10.4161/gmic.2.2.15416.
42. Pearson-Leary et al. Mol Psychiatry, 2019. doi:10.1038/s41380-019-0380-x.
43. Quirk et al. J Neurosci 20: 6225-6231, 2000. doi:10.1523/JNEUROSCI.20-16-06225.2000.
44. Riggio et al. Clin Gastroenterol Hepatol 9: 181-183, 2011. doi:10.1016/j.cgh.2010.10.002.
45. Russell et al. Mol Nutr Food Res 57: 523-535, 2013. doi:10.1002/mnfr 201200594.
46. Samala et al. Alcohol Clin Exp Res 42: 1933-1938, 2018. doi:10.1111/acer. 13862.
47. Schuppan et al. Lancet 371: 838-851, 2008. doi:10.1016/S0140-6736(08)60383-9.
48. Segata et al. Genome Biol 12: R60, 2011. doi:10.1186/gb-2011-12-6-r60.
49. Shawcross et al. Expert Rev Gastroenterol Hepatol 9: 539-542, 2015. doi:10.1586/17474124.2015.1035257.
50. Tull et al. J Anxiety Disord 24:55-62, 2010. doi:10.1016/j.janxdis.2009.08.006.
50a. US Department of Veterans Affairs. PTSD National Center for PTSD. www.ptsd.va.gov/professional/treat/essentials/dsm5_ptsd.asp#one.
51. Vilstrup et al. et al. Hepatology 60: 715-735, 2014. doi:10.1002/hep.27210.
52. Weissenborn et al. J Hepatol 34: 768-773, 2001. doi:10.1016/S0168-8278(01)00026-5.

### Example 3

### Data Collection and Analysis

When conducting metagenomics analyses as described herein, the following protocol was used.

A Clinical Plan was developed that included defining number of patients needed to optimize the power of study, and to track subjects identified as participants in the study. At least one control population that matched the general naïve population was identified. Confounding metadata (such as alcohol use, over the counter drug use, antibiotic use, diet, and Social Economic Status) was identified and tracked.

Sample Collection and Inventory was conducted by providing barcoded collection tubes and the barcode was linked to the anonymous Subject ID, sample, and metadata. In addition, a direct linkage of samples to the subject metadata was established in a relational database. Samples are checked, e.g. for contaminants (such as lubricant on rectal swabs) and rejected or approved as suitable, and suitable samples were frozen until sample extraction.

Prior to sample extraction and analysis, a Fingerprint ID was assigned to each sample with the sample's Barcode, e.g. in an Excel template. Samples were extracted following a manufacturers' protocol, via procedures that are known in the art.

Sample fingerprinting include a Quality Control step make sure each sample is linearly amplified without kinetic bias (Sikaroodi 2012, and U.S. Pat. No. 8,603,749). The following procedures were generally conducted as per manufacturer's protocol: Emulsion PCR, NextGen high-throughput sequencing and Demultiplexing. The excel template generated from the PCR sample sheet was used to produce a “MBAC Tag” file that links each Fingerprint ID with a Unique ID (Run number and the name of the primer tag). A PERL script was run to demultiplex sequence data and annotate the read ID with the sample ID, e.g. using either Unique ID or Fingerprint ID. It is noted that when Fingerprint ID is used, then duplicate runs from a sample were combined. The MBAC format for the FASTQ files had “Read ID-Unique Sequence ID” where Read ID is a unique number generated by the sequencer. It is also noted that the Qiimel format, Qiime2 and USEARCH formats are different and convertors in a Galaxy portal was used to convert data as needed. The data was distributed in Galaxy and backed up on a server.

Taxa were identified using the RDP11 Bayesian Classifier. RDP11 output was concatenated with other sequencing runs for meta-analysis, and a relative abundance matrix was created and linked to meta-data by Lefse Analysis. PERL script was run on the relative abundance matrix to create files with binary comparisons of metadata classes. Linear Discriminant Analysis with Effect Size was performed on binary relative abundance matrices.

Multivariate association with Linear Models was performed as follows: MaAsLin was run on a relative abundance matrix with multiple classes of metadata to identify associations. A Correlation Network Analysis was performed using Spearman correlations computed between all features and classes using an MBAC Galaxy portal. Correlation networks were visualized in Cytoscape. Correlation Difference Network Analysis was performed using differential Spearman correlations computed between classes using our MBAC Galaxy portal, and Correlation Difference networks were visualized in Cytoscape

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A metagenomic method of diagnosing cognitive impairment in a subject with cirrhosis, comprising
collecting a stool sample and/or a saliva sample from the subject with cirrhosis;
contacting at least a portion of the stool sample and/or at least a portion of the saliva sample with a substrate comprising nucleic acid sequences that bind to:
for the stool sample, 16S rRNA of at least one of Veillonellaceae, Enterococcus, Proteobacteria, Ruminococcaceae and Lachnospiraceae; and
for the saliva sample, 16S rRNA of at least one of Lactobacillaceae, Coriobacteriaceae, Clostridiales cluster XI, Aerococcaceae and Prevotellaceae;
detecting the presence of microbes by sequencing 16S rRNA to which the nucleic acid sequences are bound; and
diagnosing the subject as having cognitive impairment when
for the stool sample, the presence of one or more of Enterococcus, Proteobacteria and Veillonellaceae is detected; and
for the saliva sample, to the presence of one or more of the Lactobacillaceae, Coriobacteriaceae, Clostridiales cluster XI, Aerococcaceae and Prevotellaceae is detected.

2. The metagenomic method of claim 1, wherein the step of collecting is performed by collecting the stool sample.

3. The metagenomic method of claim 1, wherein the step of collecting is performed by collecting the saliva sample.

4. The metagenomic method of claim 1, wherein the nucleic acid sequences bind to 16S rRNA of Veillonellaceae, Enterococcus, Proteobacteria, Ruminococcaceae and Lachnospiraceae.

5. The metagenomic method of claim 1, wherein the nucleic acid sequences bind to 16S rRNA of Lactobacillaceae, Coriobacteriaceae, Clostridiales cluster XI, Aerococcaceae and Prevotellaceae.

6. The metagenomic method of claim 1, wherein the step of detecting is performed using one or more of the following: microarrays, qPCR and RNA sequencing.

7. The metagenomic method of claim 1, wherein the step of detecting comprises the steps of amplifying and tagging the 16S rRNA by PCR with an amplification primer pair comprising:
i) a high throughput sequencing adaptor, ii) a tag sequence to identify the originating sample, and iii) a priming sequence that is the same in each sample, the priming sequence hybridizing 3′ to a genetic region that is variable across microbial species; and
sequencing the 16S rRNA, wherein 16S rRNA sequences are assigned to the originating sample by the nucleotide sequence of the tag sequence.

8. A method of detecting and treating cognitive impairment in a subject suffering from a chronic disease, comprising
establishing a microbial signature for one or both of stool and saliva of the subject by detecting, metagenomically, the presence of at least one microbe in one or both of a stool sample and a saliva sample of the subject,
comparing the microbial signature to at least one corresponding reference microbial signature, wherein the at least one corresponding reference microbial signature is a negative reference microbial signature obtained from subjects who are not suffering from covert cognitive impairment and/or a positive reference microbial signature obtained from subjects who are suffering from covert cognitive impairment; and
treating a subject for cognitive impairment when the microbial signature differs from that of the negative reference microbial signature and/or is the same as that of the positive reference microbial signature;
wherein the step of establishing is performed by
identifying a subject having the chronic disease,
collecting a stool sample and/or a saliva sample from the subject; and
detecting microbes present in the stool sample and/or the saliva sample;
and wherein the step of detecting is performed by sequencing 16S rRNA.

9. The method of claim 8, wherein the step of treating includes administering to the subject one or more of: rifaximin, lactulose a calcineurin inhibitor (CNI), omega 3 fatty acids, and a bacteriotherapy.

10. The method of claim 9, wherein the CNI is cyclosporine or tacrolimus.

11. The method of claim 8, wherein the chronic disease is cirrhosis, heart failure, chronic kidney disease, diabetes or chronic obstructive pulmonary disease.

12. The method of claim 11, wherein the chronic disease is cirrhosis.

13. The method of claim 12, wherein the cognitive impairment is covert hepatic encephalopathy (CHE).

14. The method of claim 12, wherein the microbial signature is established by
in the stool sample, detecting the presence of at least one of Veillonellaceae and Lachnospiraceae; and/or
in the saliva sample, detecting the presence of at least one of Lactobacillaceae, Coriobacteriaceae, Clostridiales cluster XI, Aerococcaceae and Prevotellaceae.

15. The method of claim 14, wherein
the presence of Veillonellaceae in the stool sample indicates that the subject has CHE and the presence of Lachnospiraceae indicates that the subject does not have CHE; and
the presence of Lactobacillaceae and/or Coriobacteriaceae in the saliva sample indicates that the subject has CHE; and the presence of Clostridiales clusterXI, Aerococcaceae and/or Prevotellaceae indicates that the subject does not have CHE.

16. The method of claim 10, wherein the bacteriotherapy includes administration of one or more of Lachnospiraceae, Clostridiales clusterXI, Aerococcaceae and/or Prevotellaceae or agents such as phages, dietary, medication or and other methods that affect these taxa.

17. The method of claim 12, wherein the subject has PTSD.

18. The method of claim 17, wherein the microbial signature is established by, in the stool sample, detecting the presence of at least one of Enterococcus, Proteobacteria, Ruminococcaceae and Lachnospiraceae.

19. The method of claim 18, wherein
the presence of Enterococcus and/or Proteobacteria in the stool sample indicates that the subject has cognitive impairment and
the presence of Ruminococcaceae and/or Lachnospiraceae indicates that the subject does not have cognitive impairment.

20. The method of claim 16, wherein the bacteriotherapy includes administration of one or both of Ruminococcaceae and Lachnospiraceae or agents such as phages, dietary, medication or and other methods that affect these taxa.
